(19) United States
(12) Patent Application Publication
(10) Pub. No.: US 2012/0100160 A1
(43) Pub. Date: Apr. 26, 2012

(54) Methods for Inducing Mixed Chimerism

(75) Inventors: Carrie Lucas, Chevy Chase, MD (US); Megan Sykes, Bronx, NY (US); Judy Lieberman, Brookline, MA (US); Ann Schlesinger, Brookline, MA (US); Motomu Shimaoka, Mie-prefecture (JP)

(73) Assignees: IMMUNE DISEASE INSTITUTE, Boston, MA (US); THE GENERAL HOSPITAL CORPORATION, Boston, MA (US)

(21) Appl. No.: 13/131,215

(22) PCT Filed: Nov. 25, 2009
(86) PCT No.: PCT/US09/65945

§ 371 Date: Dec. 14, 2011

Related U.S. Application Data
(60) Provisional application No. 61/200,311, filed on Nov. 26, 2008.

(51) Int. Cl. A61K 39/395; A61P 37/06; A61K 35/14; B82Y 5/00

(52) U.S. Cl. 424/181.1; 424/193.1; 977/773; 977/906

(57) ABSTRACT

Fusion protein-siRNA complexes that specifically target activated T cells, and methods of use thereof, are described.

### CLAIM OF PRIORITY

This application claims the benefit of U.S. Provisional Patent Application Ser. No. 61/200,311, filed on Nov. 26, 2008, the entire contents of which are hereby incorporated by reference.

### FEDERALLY SPONSORED RESEARCH OR DEVELOPMENT

This invention was made with Government support under NHLBI R21 Grant HL094789-01 awarded by the National Institutes of Health. The Government has certain rights in the invention.

### TECHNICAL FIELD

This invention relates to methods of inducing mixed chimerism for transplant tolerance, using small interfering RNAs (siRNAs) specifically targeted to T cells.

### BACKGROUND

Solid organ transplantation is associated with a high incidence of complications due to the toxicity of chronic immunosuppressive drugs and the development of chronic rejection. The ultimate method of circumventing these obstacles would be to induce donor-specific immune tolerance. The goal of the work proposed here is to develop an approach to inducing mixed hematopoietic chimerism and donor-specific tolerance using a low dose of total body irradiation (TBI) and intravenously injected small interfering RNAs (siRNAs) that are delivered specifically to donor-reactive immune cells. Studies in preclinical models have demonstrated that establishment of mixed allogeneic hematopoietic chimerism using non-myeloablative conditioning followed by allogeneic bone marrow transplantation (BMT) can reliably induce specific tolerance to solid organ transplants with minimal toxicity (1-4). Mixed chimerism denotes coexistence of donor and recipient hematopoietic stem cells in the same individual, resulting in lifelong multilineage hematopoiesis from both sources and central tolerance of newly developing T cells recognizing recipient or donor antigens. Any subsequent organ graft from the same donor is thereby accepted without immunosuppression. Procedures for achieving mixed chimerism require a method of overcoming the pre-existing T cell immune barrier to donor marrow engraftment. Upon establishment of mixed chimerism in murine models, the recipient will accept tissue grafts from the donor with no long-term immunosuppression, no chronic rejection, and no graft-versus-host disease (GvHD). An established regimen permitting engraftment of donor hematopoietic stem cells (HSCs) in mice involves costimulation blockade using an anti-CD154 mAb (5). However, clinical use of anti-CD154 has been associated with thromboembolic complications (6-10). Thus, the development of alternative therapeutic approaches with comparable success and low toxicity will facilitate translation of this approach to the clinic.

### SUMMARY

In vivo delivery of siRNAs silencing transcripts that are critical for T cell activation, proliferation, and/or survival specifically into donor-reactive T cells is expected to result in anergy and deletion of pre-existing donor-reactive T cells when given with bone marrow transplantation. Mixed allogeneic chimerism and subsequent central deletion will assure life-long donor-specific tolerance. Thus, provided herein is a system of in vivo delivery of siRNAs directly into activated T cells to induce tolerance to grafts, e.g., allogeneic bone marrow (BM) grafts or solid organ grafts, thereby achieving mixed chimerism and subsequent central deletional tolerance.

Thus, in one aspect the invention provides methods for inducing tolerance to a tissue or cell transplant in a subject. The methods include administering to the subject (a) a composition comprising a T-cell specific siRNA delivery reagent complexed with an siRNA that specifically induces anergy and death of activated T cells; and (b) a hematopoietic stem cell transplant.

In some embodiments, the T-cell specific siRNA delivery reagent includes (i) a fusion protein for delivery of a nucleic acid to activated T cells, wherein the fusion protein includes a first portion comprising a T-cell targeting sequence that binds specifically to activated T cells; and at least a second portion comprising a cationic sequence that electrostatically binds nucleic acid molecules.

In some embodiments, the T-cell specific siRNA delivery reagent includes a nanoparticle, wherein the surface of the nanoparticle has attached thereto a T-cell targeting sequence and a cationic sequence that enables electrostatic binding of negatively charged siRNA molecules.

In some embodiments, the T-cell targeting sequence is selected from the group consisting of ICAM-1 or portions thereof, or antibodies or antigen-binding portions thereof (e.g., scFV, Fab, or Fab′2) that specifically bind to the HA conformation of LFA-1, CD69, CD25, CD44, ICOS, or an activated T-cell specific cytokine receptor.

In some embodiments, the cationic sequence that enables electrostatic binding of negatively charged siRNA molecules comprises human protamine or a cationic nucleic acid-binding portion thereof.

In some embodiments, the fusion protein further comprises a secretion signal peptide that promotes secretion from the cell.

In some embodiments, the fusion protein further comprises a multimerization domain, e.g., IgG Fc having at least an immunoglobulin CH2 and CH3 domain.

In some embodiments, the fusion protein further comprises one or more linkers between the different portions segments, e.g., a linker between the first and second portions.

In some embodiments, the fusion protein further comprises a protein purification sequence, e.g., His6 an Fc region.

In some embodiments, the siRNA specifically targets a gene encoding a protein selected from the group consisting of RasGRP1, cyclin D1, and bcl-xL include bcl-2, mcl-1, Aid, N-ras, SOS, Zap70, mTOR, NFAT, NFkB, polo-like kinases (plk), cFLIP, and ICAD.

In some embodiments, the methods also include transplanting a tissue or organ into the subject.

Also provided herein is the use a composition including (i) a fusion protein for delivery of a nucleic acid to activated T cells, wherein the fusion protein comprises a first portion comprising a T-cell targeting sequence that binds specifically to activated T cells; and at least a second portion comprising a cationic sequence that electrostatically binds nucleic acid molecules, and (ii) an siRNA that specifically induces anergy and death of activated T cells; in a method of inducing tolerance to a tissue or cell transplant in a subject.

In a further aspect, the invention provides the fusion proteins described herein, e.g., ICAM-protamine fusion proteins described herein, nucleic acids encoding those fusion proteins, vectors comprising the nucleic acids, and cells including or expressing the vectors.

The methods described herein are useful for inducing tolerance via allogeneic bone marrow transplantation. A major advantage of this approach is its versatility (i.e., ability to silence any transcript) and specificity. This approach may revolutionize the capacity to treat patients with a variety of disorders. The application of siRNA therapeutics for the purpose of inducing mixed chimerism is promising because only transient therapy is needed to remove pre-existing donor-reactive cells from the recipient. Once this is achieved, life-long central tolerance will maintain indefinite unresponsiveness to donor antigens. Another advantage is that the size of the delivery construct is large enough to escape clearance by the kidneys.

A “recipient” is a subject into whom a stem cell, tissue, or organ graft is to be transplanted, is being transplanted, or has been transplanted. An “allogeneic” cell is obtained from a different individual of the same species as the recipient and expresses “alloantigens,” which differ from antigens expressed by cells of the recipient.

A “xenogeneic” cell is obtained from a different species than the recipient and expresses “xenoantigens,” which differ from antigens expressed by cells of the recipient.

A “donor” is a subject from whom a stem cell, tissue, or organ graft has been, is being, or will be taken. “Donor antigens” are antigens expressed by the donor stem cells, tissue, or organ graft to be transplanted into the recipient. “Third party antigens” are antigens that differ from both antigens expressed by cells of the recipient, and antigens expressed by the donor stem cells, tissue, or organ graft to be transplanted into the recipient. The donor and/or third party antigens may be alloantigens or xenoantigens, depending upon the source of the graft. An allogeneic or xenogeneic cell administered to a recipient can express donor antigens, i.e., some or all of the same antigens present on the donor stem cells, tissue, or organ to be transplanted, or third party antigens. Allogeneic or xenogeneic cells can be obtained, e.g., from the donor of the stem cells, tissue, or organ graft, from one or more sources having common antigenic determinants with the donor, or from a third party having no or few antigenic determinants in common with the donor.

A “hematopoietic stem cell” is a cell, e.g., a bone marrow or a fetal liver cell, which is multipotent, e.g., capable of developing into multiple lineages, e.g., any myeloid and lymphoid lineages, and self-renewing, e.g., able to provide durable hematopoietic chimerism.

A compound that “specifically” binds to a target molecule is a compound that binds to the target molecule and does not substantially bind to other molecules.

As used herein, the term “nucleic acid molecule” includes DNA molecules (e.g., a cDNA or genomic DNA) and RNA molecules (e.g., an mRNA) and analogs of the DNA or RNA generated, e.g., by the use of nucleotide analogs. The nucleic acid molecule can be single-stranded or double-stranded, but preferably is double-stranded DNA.

The term “isolated or purified nucleic acid molecule” includes nucleic acid molecules which are separated from other nucleic acid molecules that are present in the natural source of the nucleic acid. For example, in various embodiments, the isolated nucleic acid molecule can contain less than about 0.1 kb of 5′ and/or 3′ untranslated nucleotide sequences which naturally flank the nucleic acid molecule, e.g., in the mRNA. Moreover, an “isolated” nucleic acid molecule, such as a cDNA molecule, is substantially free of other cellular material, or culture medium when produced by recombinant techniques, or substantially free of chemical precursors or other chemicals when chemically synthesized.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic diagram of an exemplary ICAM-1-Fc-Protamine construct.

FIG. 2 is a pair of images of Western blots demonstrating purified construct blotted with anti-murine ICAM-1 (left panel) and anti-protamine (right, duplicate). PD, pull down. WB, Western blot.

FIG. 3 is a bar graph showing the results of a V-bottom cell adhesion assay. 96-well V-bottom plates were coated with 10, 5, or 1 ug/ml of the ICAM-1 construct prior to addition of activated or unactivated TK1 cells or human K562 cells. WT, wild type. +Mn, activated LFA-1. −Mn, unactivated LFA-1.

FIG. 4 is an image of a Coomassie gel demonstrating dimerization of the ICAM-1 protamine construct in non-reducing conditions. The middle lane is reducing conditions, and the right lane is non-reducing conditions.

FIG. 5 is a bar graph comparing percent knockdown after delivery using the murine ICAM-1 DID2-protamine construct and the AL-57-protamine construct.

FIG. 6 is a bar graph showing the results of a flow cytometry-based multimer binding assay on primary murine splenocytes that were unstimulated (PBS, open bars) or stimulated with Mg and EGTA (filled bars).

FIG. 7A is a bar graph showing the results of a flow cytometry-based multimer binding assay using multimerized fusion protein plus anti-Fc Fab′2 APC or non-multimerized fusion protein with anti-Fc Fab′2 APC as a secondary.

FIG. 7B shows the flow cytometry histogram from which the data in FIG. 7A was obtained. Light grey filled area, Anti-Fc alone. Dark grey line, multimerized. Medium grey line, non-multimerized.

FIG. 8A is a set of six scatter plots showing the expression of CD45 and CD11a in EL4 (left column of panels), TK-1 (middle column), and DC2.4 (right column); both are expressed only in the TK-1 cells.

FIG. 8B is a bar graph showing that binding of the multimer can be blocked by pre-incubation of the cells with anti-CD11a to block LFA-1.

FIG. 8C shows two flow cytometry histograms from which the data in FIG. 8B was obtained. Light grey filled area, multimerized. Dark grey line, multimerized plus anti-CD11a blocking antibody.

FIGS. 9A-9B are bar graphs showing the results of CD45 knockdown in TK-1 cells using 2×10⁵ cells, 80 pmoles ICAM-1 fusion protein, 10 pmoles anti-Fc Fab′2, and various amounts of CD45 siRNA to obtain a 2:1, 4:1, or 6:1 ratio of siRNA to fusion protein, measured by median fluorescent intensity (9A) and percent knockdown (9B).

FIG. 10 is a bar graph showing the results of CD45 knockdown in TK-1 cells using 2×105 cells, 80 pmoles ICAM-1 fusion protein, 10 pmoles anti-Fc Fab′2, and various amounts of CD45 siRNA to obtain a 4:1, 6:1, 8:1, 10:1, or 12:1 ratio of siRNA to fusion protein, measured by median fluorescent intensity. Cells were cultured with 2-3% FCS.

### DETAILED DESCRIPTION

One obstacle to in vivo manipulation of gene expression using siRNAs is delivery into specific cell types, and delivery into difficult-to-transfect lymphocytes is a special challenge. Methods have been described to introduce siRNAs into human lymphocytes or specifically into only activated lymphocytes by mixing siRNAs with a fusion protein composed of an antibody fragment recognizing the human beta2 integrin lymphocyte function-associated antigen-1 (LFA-1) expressed on all leukocytes (or just the high affinity (HA) form of LFA-1 on activated leukocytes) linked to an siRNA-binding protamine peptide (11;12). Provided herein are methods to induce mixed chimerism by introducing siRNAs specifically into activated lymphocytes, using an siRNA delivery reagent consisting of domains 1 and 2 of murine intercellular adhesion molecule-1 (ICAM-1), a major ligand of LFA-1, fused to the same protamine peptide. This delivery reagent binds specifically to activated leukocytes, which express the HA conformation LFA-1, resulting in internalization of electrostatically conjugated siRNAs. As demonstrated herein, the siRNA-ICAM-1 fusion protein complex binds specifically to activated lymphocytes of both mice and humans. This construct can be used to target siRNAs to recipient T cells recognizing donor alloantigens expressed on a bone marrow graft.

These complexes provide a therapeutic approach to inducing tolerance to bone marrow grafts using non-myeloablative conditioning. Establishing mixed chimerism and donor-specific tolerance in patients can be used not only to promote acceptance of any solid organ graft without immunosuppressive therapy, but also to treat hematologic disorders such as hemoglobinopathies, as well as inborn errors of metabolism (13-15) and potentially autoimmune diseases (16-18). This cell type-specific siRNA-based approach can also be used in the treatment of many other diseases, including chronic viral infections (12;19). siRNAs for use in the present methods can be designed to silence any transcript of interest and can be screened in cell culture for those that are highly specific with limited off-target effects (20;21).

The ultimate goal in transplantation is donor-specific tolerance that is robust and long-lasting. This has been achieved in both murine models and clinical protocols involving bone marrow transplants (BMT) between genetically disparate individuals (i.e., allogeneic individuals) (2;5;22-24). Two major obstacles must be overcome in order to achieve allogeneic bone marrow engraftment. One is competition with the recipient hematopoietic system in the bone marrow niche. This can be overcome by relatively mild myelosuppressive treatments, such as low-dose total-body irradiation (TBI), or by giving very high numbers of donor hematopoietic cells (25-28). The second obstacle is T cell-mediated immune resistance, which can be overcome by either global depletion of mature T cells in the periphery and the thymus (1) or by tolerance induction with costimulation blockade (4). Upon acceptance of the bone marrow graft, central (intra-thymic) tolerance of any newly-arising T cells is assured due to the presence of APCs originating from donor and recipient hematopoietic stem cells (HSCs) present in the recipient bone marrow (29). Once mixed chimerism is established, newly developing T cells differentiating from both the recipient HSCs and the engrafted donor HSCs undergo negative selection in the host thymus via interactions with host- and donor-type dendritic cells (DCs), respectively (30). Permanent coexistence of host and donor HSCs allows for a continued supply of DCs that induce life-long, mutual tolerance of host and donor grafts. As such, mixed chimeras demonstrate specific acceptance of donor but not third party skin grafts (followed for greater than 100 days) placed any time after BMT and do not develop any GvHD (31;32). Reliably translating this approach into the clinic will improve the long-term health of transplant patients by obviating the need for long-term immunosuppression, which will markedly reduce the high risks of opportunistic infection, malignancy, hypertension, metabolic disorders, and other associated toxicities. Moreover, the achievement of systemic tolerance would overcome the problem of late graft loss due to chronic rejection, a limitation to the success of transplantation that has not been ameliorated by recent advances in immunosuppressive therapies.

Combined bone marrow and renal allotransplantation has been used successfully in large animal models (33) and, most recently, in small groups of patients with renal failure due to multiple myeloma and in patients with no malignant disease (22-24;34). However, the non-human primates and the latter group of patients, who received transplants from extensively HLA-mismatched, related, haploidentical donors, did not have durable, long-lasting mixed chimerism. Nonetheless, transient mixed chimerism surprisingly enabled long-term acceptance of the kidney allograft with no sustained immunosuppression, and the unacceptable complication of graft-versus-host disease (GVHD) did not occur. The regimens used to establish mixed chimerism in these patients, however, involved extensive T cell depletion of the recipients, leaving them significantly immunosuppressed for many weeks due to slow regeneration of T cells in the adult thymus. While these results provide important proof of principle for the potential of mixed chimerism to achieve transplant tolerance, the mechanisms of the long-term tolerance achieved by transient mixed chimerism in these patients are clearly more complex than the central deletion described above for the murine model, in which chimerism is life-long. Evidence suggests that the kidney allograft itself plays an important role in this tolerance process in the monkey model (35) and in these patients (22-24) (and our unpublished data). However, other types of grafts, such as the heart, are more immunogenic than kidneys in large animals (36-38) and probably in humans. A non-toxic approach to achieving durable, life-long mixed chimerism, and, therefore, systemic tolerance, would permit the acceptance of any type of graft from the same donor, including heart, pancreatic islets, liver, pancreas, and intestine. The challenge for scientists developing hematopoietic cell transplantation (HCT) as an approach to clinical transplantation tolerance is to establish regimens that permit durable mixed chimerism across HLA barriers without GvHD and with minimal conditioning-associated toxicity and minimal immunosuppression.

A novel and theoretically promising strategy involves specific delivery of small interfering (si)RNAs that silence transcripts required for sustained T cell activation and survival into activated T cells via an activation-dependent cell surface protein. This approach is expected to result in rapid unresponsiveness and deletion of pre-existing donor-reactive T cells following allogeneic BMT, enabling bone marrow and thymic engraftment by donor cells, resulting in central tolerance and long-term multilineage mixed chimerism. SiRNAs for this purpose will be used only transiently, specifically deleting donor-reactive T cells until central tolerance takes effect.

According to the methods and compositions described herein, delivery of the siRNAs will be achieved with a reagent produced by fusing a cationic sequence, e.g., from human protamine, to a protein (or fragment thereof) that binds a specific cell surface antigen (11;12). Such proteins (or fragments) may include single chain variable fragments (scFvs) of an antibody, the Fab fragment of an antibody, or the binding domains of cell surface receptor ligands. Incubation of the cationic fusion protein with negatively charged siRNAs enables formation of a charge-dependent complex containing roughly 6 siRNAs per complex (12).

T-Cell Specific Delivery Reagents

The methods described herein include the use of T-cell specific reagents to deliver siRNA to T cells. Suitable reagents include fusion proteins that include a T-cell targeting sequence and a cationic sequence for binding of the nucleic acids, as well as surface-modified nanoparticles that include a T-cell targeting moiety and an siRNA or an siRNA-binding moiety, e.g., a cationic sequence such as protamine that enables electrostatic binding of negatively charged siRNA molecules. See, e.g., Weyermann et al., Eur. J. Pharma. Biopharm. 59:431-438 (2005); Yuan et al., J. Nanosci. Nanotechnol. 6(9-10):2821-2828 (2006); Katas and Alpar, J. Contr. Rel. 115(2):216-225 (2006); Zillies and Coester, 2004 International Conference on MEMS, NANO and Smart Systems (ICMENS'04), Abst 432 (2004); Lambert et al., Drug Deliv. Rev. 47(1):99-112 (2001) (describes nucleic acids loaded to polyalkyl-cyanoacrylate (PACA) nanoparticles); Fattal et al., J. Contr. Rel. 53(1-3):137-43 (1998) (describes nucleic acids bound to nanoparticles); Schwab et al., Ann. Oncol. 5 Suppl. 4:55-8 (1994) (describes nucleic acids linked to intercalating agents, hydrophobic groups, polycations or PACA nanoparticles); and Godard et al., Eur. J. Biochem. 232(2):404-10 (1995) (describes nucleic acids linked to nanoparticles).

Fusion Proteins for Delivery of siRNA

In general, the fusion proteins useful for delivering siRNAs into activated, donor-reactive T cells, the fusion proteins include the following components:

1. An optional secretion signal peptide, that promotes secretion from the cell. An exemplary sequence is MASTRAKPTLPLLLALVTVVIPG (SEQ ID NO:1)). Other exemplary sequences include MRRRSLLILV (SEQ ID NO:2) and MRRRRSLLILV (SEQ ID NO:3) (see Tsuchiya et al., Nucleic Acids Research Suppl. No. 3:261-262 (2003)); others are known in the art, see, e.g., Kaiser et al., Science, 235(4786):312-317 (1987); Barash et al., Biochem. Biophys. Res. Comm. 294(4):835-842 (2002); Sperandio, Trends Microbiol., 8(9):395 (2000); Sletta et al., Appl Environ Microbiol. 73(3):906-912 (2007); EP0266057; and U.S. Pat. Nos. 6,733,997 and 7,071,172. A secretion signal sequence can be identified and selected from a database, e.g., SPdb (Choo et al., BMC Bioinformatics 6:249 (2005)), which lists 2512 experimentally verified signal sequences. In general, a signal sequence should be selected that induces secretion of the fusion protein from the type of cells in which the fusion protein is produced.

2. A T-cell targeting sequence, i.e., a sequence that encodes a protein that binds specifically to activated T cells. Examples include ICAM-1 or portions thereof, or ligands or antibodies or antigen-binding portions thereof that specifically bind to the HA conformation of LFA-1, CD69, CD25, CD44, ICOS, or an activated T-cell specific cytokine receptor. In some embodiments, a mAb against a T-cell specific marker, e.g., the HA conformation of LFA-1, or an antigen-binding portion thereof, e.g., an Fab, Fab′2, or scFv, can be used (62).

Full names of these exemplary T cell targeting sequences and genbank accession numbers are given in Table 1.

TABLE 1
T cell targeting proteins

| | | |
|---|---|---|
| ICAM-1 | Homo sapiens intercellular adhesion | NM_000201.2 |
| | molecule 1 (ICAM1) | |
| CD69 | Homo sapiens CD69 molecule (CD69), | NM_001781.2 |
| | transcript variant 1 | |
| CD25 | Homo sapiens interleukin 2 receptor, | NM_000417.1 |
| | alpha (IL2RA) | |
| CD44 | Homo sapiens CD44 molecule (Indian | NM_000610.3 |
| | blood group) (CD44), transcript | |
| | variant 1 | |
| | Homo sapiens CD44 molecule (Indian | NM_001001389.1 |
| | blood group) (CD44), transcript | |
| | variant 2 | |
| | Homo sapiens CD44 molecule (Indian | NM_001001390.1 |
| | blood group) (CD44), transcript | |
| | variant 3 | |
| | Homo sapiens CD44 molecule (Indian | NM_001001391.1 |
| | blood group) (CD44), transcript | |
| | variant 4 | |
| | Homo sapiens CD44 molecule (Indian | NM_001001392.1 |
| | blood group) (CD44), transcript | |
| | variant 5 | |
| ICOS | Homo sapiens inducible T-cell | NM_012092.2 |
| | co-stimulator (ICOS) | |

3. An optional multimerization domain. The term “multimerization domain” includes any polypeptide that forms a dimer (or higher order complex, such as a trimer, tetramer, etc.) with another polypeptide. Optionally, the multimerization domain associates with other, identical multimerization domains, thereby forming homomultimers. An IgG Fc element is an example of a multimerization domain that tends to form homomultimers, e.g., an Fc having at least an immunoglobulin CH2 and CH3 domain. The CH2 and CH3 domains can form at least a part of the multimerization domain of the protein molecule (e.g., antibody) when functionally linked to a dimerizing or multimerizing domain such as the antibody hinge domain. The Fc domains are preferably derived from human germline sequences such as those disclosed in WO2005005604. In general, multimerization domains will be used when the T cell targeting sequence functions more efficiently when dimerized or multimerized; for example, a multimerization domain is desirable when the T-cell targeting sequence is ICAM.

4. An optional linker. Linkers useful in the present compositions are generally flexible and must not interfere with the functions of any of the other components. Linkers of 1, 2, 3, 4, 5, 6, 7, 8, 9, 10, or more amino acids can be used. In a preferred embodiment, the linker includes alanine and guanine residues.

5. A cationic sequence that enables electrostatic binding of negatively charged siRNA molecules. Examples include, e.g., human protamine or a portion thereof, e.g., amino acids 8 through 29 of human protamine (RSQSRSRYYRQRQRSRRRRRRS (SEQ ID NO:4).

6. An optional protein purification sequence, e.g., His6 or Fc, that facilitates purification of the fusion protein. In some embodiments, an Fc region is included in the fusion protein and acts as both a multimerization domain and as a purification sequence (purification of Fc-containing fusion proteins can be achieved using protein A, e.g., bound to a substrate such as a bead or solid surface, e.g., in a column).

These segments can be in no specific order or in the order from N to C terminus as set forth above.

In one example of the present compositions, the ICAM-1-LFA-1 interaction is exploited. LFA-1, or αLβ2 integrin, is expressed constitutively on all T cells, B cells, NK cells, monocytes, macrophages, dendritic cells, and neutrophils (68-71). The integrin a subunit contains an inserted (I) domain, which contains a metal ion-dependent adhesion site (MIDAS) (72). Upon addition of manganese (Mn2+) (or, alternatively, Mg2+ and EGTA) to cells expressing LFA-1, the MIDAS in the I domain becomes occupied, converting LFA-1 to an open conformation by displacing the C terminal helix (73). This allows for a convenient method of conversion of LFA-1 to its HA state for in vitro binding assays. Physiologically, LFA-1 can be activated to convert to its HA state through inside-out signaling that occurs when an extracellular activation signal is transduced into the cell, resulting in talin binding to the cytoplasmic domain of the β subunit of LFA-1 (74-78). This dissociates the salt bridge linking the cytoplasmic tails of the α and β subunits and propagates the membrane-proximal conformational change to the extracellular domains, resulting in exposure of the I domain for ligand binding. The physiological ligands for LFA-1 include ICAM-1, ICAM-2, and ICAM-3. ICAM-1 (CD54) is expressed on endothelial cells, lymphocytes, epithelial cells, and fibroblasts and can bind not only to LFA-1 but also to Mac-1, fibrinogen, and p150, 95 (79-81). Binding of ICAM-1 to LFA-1 is restricted to the HA LFA-1 conformation. This interaction promotes extravasation of activated leukocytes through post-capillary venules as well as T cell-APC adhesion and provides costimulation. LFA-1 transiently converts to its HA conformation on T cells after activation through inside-out signaling and clustering, and this conversion promotes firm adhesion to ICAM-1 (82;83).

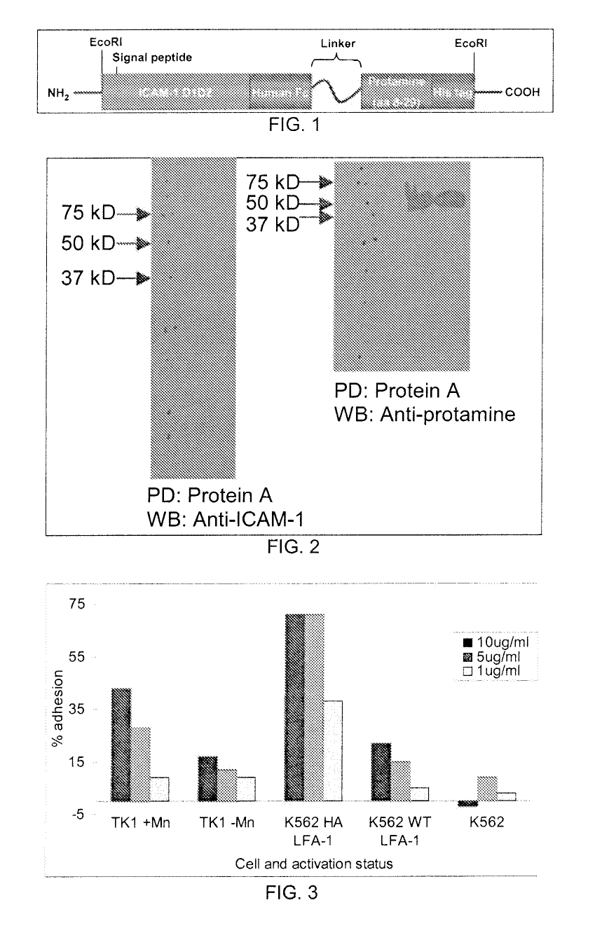

Thus, in one aspect the invention provides a fusion protein as shown in FIG. 1, containing a portion of mouse ICAM-1 that confers HA LFA-1 specificity, namely domain 1 (D1) and domain 2 (D2), permits delivery of siRNAs only to HA LFA-1-expressing cells and not to cells expressing Mac-1 or other ICAM-1 ligands. The ICAM-1 region used is predicted to bind both the human and mouse proteins (11;84). The conserved Kozak sequence (GCCACCAUGG) for ribosome binding and translation initiation was fused to D1 and D2 of murine ICAM-1, which was subsequently fused with a portion of human IgG Fc (C_{H}2 and C_{H}3), a flexible linker (GGGS). The sequence is fused to a cationic sequence, e.g., amino acids 8 through 29 of His6-tagged human protamine, enabling electrostatic binding of negatively charged siRNA molecules. In some embodiments, a secretion signal peptide (e.g., sequence: MASTRAKPTLPLLLALVTVVIPG (SEQ ID NO:1)) is included, e.g., in exon 1 of ICAM-1, to promote secretion of the fusion protein to allow for easier isolation from cells expressing the fusion protein. In some embodiments, an Fc region is included to facilitate ICAM-1 dimerization, which increases avidity for HA LFA-1, and enables pull-down of the fusion protein using protein A agarose beads.

Also provided herein are nucleic acids encoding the fusion proteins, vectors comprising the nucleic acids, and host cells comprising and/or expressing the nucleic acids and vectors.

In one embodiment, an isolated nucleic acid molecule is provided that includes a nucleotide sequence that encodes a fusion protein that is at least about 90% or more identical to the entire length of the ICAM-protamine fusion protein sequence shown in Example 1 as SEQ ID NO:7. In some embodiments, the sequence is at least about 91%, 92%, 93%, 94%, 95%, 96%, 97%, 98%, 99% or 100% to SEQ ID NO:7.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

For purposes of the present invention, the comparison of sequences and determination of percent identity between two sequences can be accomplished using a Blossum 62 scoring matrix with a gap penalty of 12, a gap extend penalty of 4, and a frameshift gap penalty of 5.

Also provided herein are vectors, preferably expression vectors, containing a nucleic acid encoding a fusion protein as described herein. As used herein, the term “vector” refers to a nucleic acid molecule capable of transporting another nucleic acid to which it has been linked and can include a plasmid, cosmid or viral vector. The vector can be capable of autonomous replication or it can integrate into a host DNA. Viral vectors include, e.g., replication defective retroviruses, adenoviruses and adeno-associated viruses.

A vector can include a nucleic acid encoding a fusion protein in a form suitable for expression of the nucleic acid in a host cell. Preferably the recombinant expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those which direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vectors of the invention can be introduced into host cells to thereby produce fusion proteins as described herein.

The recombinant expression vectors of the invention can be designed for expression of the fusion proteins described herein in prokaryotic or eukaryotic cells. For example, polypeptides of the invention can be expressed in E. coli, insect cells (e.g., using baculovirus expression vectors), yeast cells or mammalian cells. Suitable host cells are discussed further in Goeddel, (1990) Gene Expression Technology: Methods in Enzymology 185, Academic Press, San Diego, Calif. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRITS (Pharmacia, Piscataway, N.J.) which fuse glutathione S-transferase (GST), maltose E binding protein, or protein A, respectively, to the target recombinant protein.

To maximize recombinant protein expression in E. coli is to express the protein in a host bacteria with an impaired capacity to proteolytically cleave the recombinant protein (Gottesman, S., (1990) Gene Expression Technology: Methods in Enzymology 185, Academic Press, San Diego, Calif. 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

The expression vector can be a yeast expression vector, a vector for expression in insect cells, e.g., a baculovirus expression vector or a vector suitable for expression in mammalian cells.

When used in mammalian cells, the expression vector's control functions are often provided by viral regulatory elements. For example, commonly used promoters are derived from polyoma, Adenovirus 2, cytomegalovirus and Simian Virus 40.

In another embodiment, the recombinant mammalian expression vector is capable of directing expression of the nucleic acid preferentially in a particular cell type (e.g., tissue-specific regulatory elements are used to express the nucleic acid). Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example, the murine hox promoters (Kessel and Gruss (1990) Science 249:374-379) and the alpha-fetoprotein promoter (Campes and Tilghman (1989) Genes Dev. 3:537-546).

The invention further provides a recombinant expression vector comprising a DNA molecule of the invention cloned into the expression vector in an antisense orientation. Regulatory sequences (e.g., viral promoters and/or enhancers) operatively linked to a nucleic acid cloned in the antisense orientation can be chosen which direct the constitutive, tissue specific or cell type specific expression of antisense RNA in a variety of cell types. The antisense expression vector can be in the form of a recombinant plasmid, phagemid or attenuated virus. For a discussion of the regulation of gene expression using antisense genes see Weintraub, H. et al., (1986) Antisense RNA as a molecular tool for genetic analysis, Reviews—Trends in Genetics 1:1.

Also provided herein are host cells that include a nucleic acid molecule described herein, e.g., a nucleic acid molecule encoding a fusion protein within a recombinant expression vector or a nucleic acid molecule containing sequences which allow it to homologously recombine into a specific site of the host cell's genome. The terms “host cell” and “recombinant host cell” are used interchangeably herein. Such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a fusion protein as described herein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation.

A host cell of the invention can be used to produce (i.e., express) a fusion protein as described herein. Accordingly, the invention further provides methods for producing a fusion protein using the host cells described herein. In one embodiment, the method includes culturing the host cell of the invention (into which a recombinant expression vector encoding a fusion protein as described herein has been introduced) in a suitable medium such that the fusion protein is produced. In another embodiment, the method further includes isolating the fusion protein from the medium or from the host cell.

In another aspect, the invention features, a human cell, e.g., a hematopoietic stem cell, transformed with nucleic acid which encodes a fusion protein as described herein.

One advantage of some embodiments of the presently described compositions is that murine ICAM-1 binds both murine and human HA LFA-1 (11;84). Therefore, even the murine reagent could move from murine models to human trials with no or minimal modifications.

Lethality siRNAs

The compositions and methods described herein include “lethality siRNAs” to reduce proliferation and survival of T cells. For example, lethality siRNAs can target RasGRP1, cyclin D1, Hsp90, survivin, Plk1, bcl-xL, bcl-2, mcl-1, Aid, N-ras, SOS, Zap70, mTOR, NFAT, NFkB, polo-like kinases (plk), cFLIP, ICAD, survivin, and/or several other proteins involved in T cell activation and survival. Full names of these exemplary lethality genes, and GenBank Accession Nos. therefor, are given in Table 2.

TABLE 2
T-Cell Lethality Genes

| | | |
|---|---|---|
| RasGRP1 | RAS guanyl releasing protein 1 | NM_005739.3 |
| cyclin D1 | cyclin D1 | NM_053056.2 |
| Hsp90 | Homo sapiens heat shock protein 90 kDa alpha | NM_007355.2 |
| | (cytosolic), class B member 1 (HSP90AB1) | |
| survivin | Homo sapiens baculoviral IAP repeat-containing 5 | NM_001012271.1 |
| | (BIRC5), transcript variant 3 | |
| | Homo sapiens baculoviral IAP repeat-containing 5 | NM_001168.2 |
| | (BIRC5), transcript variant 1 | |
| | Homo sapiens baculoviral IAP repeat-containing 5 | NM_001012270.1 |
| | (BIRC5), transcript variant 2 | |
| Plk1 | Homo sapiens polo-like kinase 1 | NM_005030.3 |
| bcl-xL | Homo sapiens BCL2-like 1 (BCL2L1), nuclear gene | NM_138578.1 |
| | encoding mitochondrial protein, transcript variant 1 | |
| bcl-2 | Homo sapiens B-cell CLL/lymphoma 2 (BCL2), | NM_000633.2 |
| | nuclear gene encoding mitochondrial protein, | |
| | transcript variant alpha | |
| | Homo sapiens B-cell CLL/lymphoma 2 (BCL2), | NM_000657.2 |
| | nuclear gene encoding mitochondrial protein, | |
| | transcript variant beta | |
| mcl-1 | Homo sapiens myeloid cell leukemia sequence 1 | NM_021960.3 |
| | (BCL2-related) (MCL1), transcript variant 1 | |
| | Homo sapiens myeloid cell leukemia sequence 1 | NM_182763.1 |
| | (BCL2-related) (MCL1), transcript variant 2 | |
| Akt1 | Homo sapiens v-akt murine thymoma viral oncogene | NM_005163.2 |
| | homolog 1 (AKT1), transcript variant 1 | |
| | transcript variant 2 | NM_001014432.1 |
| | transcript variant 3 | NM_001014431.1 |
| N-ras | Homo sapiens neuroblastoma RAS viral (v-ras) | NM_002524.3 |
| | oncogene homolog (NRAS) | |
| SOS | Homo sapiens son of sevenless homolog 1 | NM_005633.3 |
| | (Drosophila) (SOS1) | |
| Zap70 | Homo sapiens zeta-chain (TCR) associated protein | NM_001079.3 |
| | kinase 70 kDa (ZAP70), transcript variant 1 | |
| mTOR | Homo sapiens mechanistic target of rapamycin | NM_004958.3 |
| | (serine-threonine kinase) (MTOR) | |
| NFAT1 | Homo sapiens nuclear factor of activated T-cells, | NM_001136021.1 |
| | cytoplasmic, calcineurin-dependent 2 (NFATC2), | |
| | transcript variant D | |
| | Homo sapiens nuclear factor of activated T-cells, | NM_173091.2 |
| | cytoplasmic calcineurin-dependent 2 (NFATC2), | |
| | transcript variant 2 | |
| | Homo sapiens nuclear factor of activated T-cells, | NM_012340.3 |
| | cytoplasmic, calcineurin-dependent 2 (NFATC2), | |
| | transcript variant 1 | |
| NFAT2 | Homo sapiens nuclear factor of activated T-cells, | NM_006162.3 |
| | cytoplasmic, calcineurin-dependent 1 (NFATC1), | |
| | transcript variant 2 | |
| | Homo sapiens nuclear factor of activated T-cells, | NM_172388.1 |
| | cytoplasmic, calcineurin-dependent 1 (NFATC1), | |
| | transcript variant 4 | |
| | Homo sapiens nuclear factor of activated T-cells, | NM_172390.1 |
| | cytoplasmic, calcineurin-dependent 1 (NFATC1), | |
| | transcript variant 1 | |
| | Homo sapiens nuclear factor of activated T-cells, | NM_172387.1 |
| | cytoplasmic, calcineurin-dependent 1 (NFATC1), | |
| | transcript variant 3 | |
| | Homo sapiens nuclear factor of activated T-cells, | NM_172389.1 |
| | cytoplasmic, calcineurin-dependent 1 (NFATC1), | |
| | transcript variant 5 | |
| NFkB | Homo sapiens nuclear factor of kappa light | NM_003998.2 |
| | polypeptide gene enhancer in B-cells 1 (NFKB1) | |
| RelA | Homo sapiens v-rel reticuloendotheliosis viral | NM_021975.3 |
| | oncogene homolog A (avian) (RELA), transcript | |
| | variant 1 | |
| | Homo sapiens v-rel reticuloendotheliosis viral | NM_001145138.1 |
| | oncogene homolog A (avian) (RELA), transcript | |
| | variant 2 | |
| PLK1 | Homo sapiens polo-like kinase 1 (Drosophila) | NM_005030.3 |
| | (PLK1) | |
| PLK2 | Homo sapiens polo-like kinase 2 (Drosophila) | NM_006622.2 |
| | (PLK2) | |
| c-FLIP | Homo sapiens CASP8 and FADD-like apoptosis | NM_003879.4 |
| | regulator (CFLAR), transcript variant 1 | |
| | Homo sapiens CASP8 and FADD-like apoptosis | NM_001127183.1 |
| | regulator (CFLAR), transcript variant 2 | |
| | Homo sapiens CASP8 and FADD-like apoptosis | NM_001127184.1 |
| | regulator (CFLAR), transcript variant 3 | |
| ICAD (inhibitor of | Homo sapiens DNA fragmentation factor, 45 kDa, | NM_004401.2 |
| caspase-activated | alpha polypeptide (DFFA), transcript variant 1 | |
| deoxyribonuclease) | Homo sapiens DNA fragmentation factor, 45 kDa, | NM_213566.1 |
| | alpha polypeptide (DFFA), transcript variant 2 | |

The lethality targets are selected based on evidence that their absence will result in unresponsiveness and apoptosis of T cells. Lethality siRNAs can be selected and verified by testing the ability of a candidate lethality siRNAs to induce chimerism and deletion of donor-reactive T cells in vivo. For example, ras guanine nucleotide releasing protein 1 (RasGRP1) is a guanine nucleotide exchange factor that relocates from the cytosol to the plasma membrane in a diacylglycerol-dependent manner following TCR stimulation. At the plasma membrane, RasGRP1 is in close vicinity to Ras and, therefore, is able to convert Ras from its GDP-bound state to its GTP-bound state. GTP-bound Ras is subsequently able to bind and activate effector proteins that culminate in activation of the mitogen activated protein (MAP) kinase effector pathway that controls IL-2 production and proliferation (95-97). Mice lacking RasGrp1 are immunodeficient due to disrupted TCR signaling and impaired positive but not negative selection of thymocytes (98). Reduction of cyclin D1 protein levels is expected to inhibit progression through the cell cycle and, therefore, prevent expansion and promote deletion of donor-reactive cells (11;99). Bcl-xL is an anti-apoptotic protein that functions by suppressing Bax- and Bak-mediated activation of the intrinsic pathway of cell death. Silencing of bcl-xL will induce a pro-apoptotic state in activated T cells and, therefore, lower the threshold for cell death of donor-reactive T cells (100).

Designing and Selecting Lethality siRNA Molecules

RNAi is a remarkably efficient process whereby double-stranded RNA (dsRNA, alse referred to herein as si RNAs or ds siRNAs, for double-stranded small interfering RNAs) induces the sequence-specific degradation of homologous mRNA in animals and plant cells (Hutvagner and Zamore, Curr. Opin. Genet. Dev.:12, 225-232 (2002); Sharp, Genes Dev., 15:485-490 (2001)). In mammalian cells, RNAi can be triggered by 21-nucleotide (nt) duplexes of small interfering RNA (siRNA) (Chiu et al., Mol. Cell. 10:549-561 (2002); Elbashir et al., Nature 411:494-498 (2001)), or by micro-RNAs (miRNA), functional small-hairpin RNA (shRNA), or other dsRNAs which are expressed in vivo using DNA templates with RNA polymerase III promoters (Zeng et al., Mol. Cell. 9:1327-1333 (2002); Paddison et al., Genes Dev. 16:948-958 (2002); Lee et al., Nature Biotechnol. 20:500-505 (2002); Paul et al., Nature Biotechnol. 20:505-508 (2002); Tuschl, T., Nature Biotechnol. 20:440-448 (2002); Yu et al., Proc. Natl. Acad. Sci. USA 99(9):6047-6052 (2002); McManus et al., RNA 8:842-850 (2002); Sui et al., Proc. Natl. Acad. Sci. USA 99(6):5515-5520 (2002).)

In general, the methods described herein can use dsRNA molecules comprising 16-30, e.g., 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the mRNA, and the other strand is complementary to the first strand. In some embodiments, the siRNA molecule is a 21-25 base-pair, double-stranded sequence of RNA designed with complementarity to any mRNA transcript to be silenced. The dsRNA molecules can be chemically synthesized, or can transcribed be in vitro from a DNA template, or in vivo from, e.g., shRNA. The dsRNA molecules can be designed using any method known in the art. Negative control siRNAs should have the same nucleotide composition as the selected siRNA, but without significant sequence complementarity to the appropriate genome. Such negative controls can be designed by randomly scrambling the nucleotide sequence of the selected siRNA; a homology search can be performed to ensure that the negative control lacks homology to any other gene in the appropriate genome. In addition, negative control siRNAs can be designed by introducing one or more base mismatches into the sequence.

An siRNA of the invention can be constructed using chemical synthesis and enzymatic ligation reactions using procedures known in the art. For example, an siRNA can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The siRNA can be produced biologically using an expression vector into which a nucleic acid has been subcloned in an antisense orientation.

Based upon the sequences disclosed herein, one of skill in the art can easily choose and synthesize any of a number of appropriate siRNA molecules for use in accordance with the present invention. For example, a “gene walk” comprising a series of oligonucleotides of 16-30 nucleotides spanning the length of a target nucleic acid can be prepared, followed by testing for inhibition of target gene expression. Optionally, gaps of 5-10 nucleotides can be left between the oligonucleotides to reduce the number of oligonucleotides synthesized and tested. In silico methods as known in the art and described herein can also be used to select appropriate sequences.

The methods described herein can use both siRNA and modified siRNA derivatives, e.g., siRNAs modified to alter a property such as the pharmacokinetics of the composition, for example, to increase half-life in the body, e.g., crosslinked siRNAs. Thus, the invention includes methods of administering siRNA derivatives that include siRNA having two complementary strands of nucleic acid, such that the two strands are crosslinked. In some embodiments, the siRNA derivative has at its 3′ terminus a biotin molecule (e.g., a photocleavable biotin), a peptide (e.g., a Tat peptide), a nanoparticle, a peptidomimetic, organic compounds (e.g., a dye such as a fluorescent dye), or dendrimer. Modifying SiRNA derivatives in this way may improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

Specific modifications can be introduced into the synthetic siRNA molecules to improve stability and loading into RNA-induced silencing complexes (RISCs). For example, introduction of a phosphorothioate (P═S) backbone linkage at the 3′ end protects against exonucleases, and a 2′-sugar modification, such as 2′-O-methyl or 2′-fluoro, protects against endonucleases. To improve loading into RISC, the double-stranded siRNA molecule can be designed with a mismatch at the 5′ end of the strand intended to be the active strand that binds the complementary mRNA transcript. This works because the strand with the weakest binding at the 5′ end is the one that favors binding in the deep pocket of RISC (20). Inclusion of 2′-O-methyl nucleosides into the second position of one strand of the siRNA molecules completely abrogates immune stimulation by synthetic siRNAs (48). A similar chemical modification also almost completely eliminates off-target silencing of genes containing partially homologous sequences without compromising silencing of the intended target gene.

As one of skill in the art will appreciate, the present methods and compositions can make use of antisense or other inhibitory nucleic acids in place of or in addition to siRNAs. Methods for making and using antisense molecules are known in the art.

Pharmaceutical Compositions and Methods of Administration

The methods described herein include the manufacture and use of pharmaceutical compositions, which include a fusion protein-siRNA complex as described herein that specifically targets activated T cells as active ingredients. Also included are the pharmaceutical compositions themselves.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intravenous, intradermal, and subcutaneous administration.

Methods of formulating suitable pharmaceutical compositions are known in the art, see, e.g., the books in the series Drugs and the Pharmaceutical Sciences: a Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) and microencapsulation can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

Hematopoietic Stem Cell Transplant

The present methods include the administration of a hematopoietic stem cell graft to the recipient. In some embodiments, the stem cells are, or are derived from, bone marrow. As noted above, hematopoietic stem cells are cells, e.g., bone marrow or fetal liver cells, which are multipotent, e.g., capable of developing into multiple or all myeloid and lymphoid lineages, and self-renewing, e.g., able to provide durable hematopoietic chimerism. Purified preparations of hematopoietic cells or mixed preparations, such as bone marrow, which include other cell types, can be used in the methods described herein. The preparation typically includes immature cells, i.e., undifferentiated hematopoietic stem cells; a substantially pure preparation of stem cells can be administered, or a complex preparation including other cell types can be administered. As one example, in the case of bone marrow stem cells, the stem cells can be separated out to form a pure preparation, or a complex bone marrow sample including stem cells can be used as a mixed preparation. Hematopoietic stem cells can be from fetal, neonatal, immature, or mature animals. Methods for the preparation and administration of hematopoietic stem cell transplants are known in the art, e.g., as described in U.S. Pat. Nos. 6,514,513 and 6, 208,957. For example, stem cells can be derived from peripheral blood (Burt et al., Blood, 92:3505-3514, 1998), cord blood (Broxmeyer et al., Proc. Nat. Acad. Sci. U.S.A., 86:3828-3832, 1989), bone marrow (Bensinger et al., New Eng. J. Med., 344:175-181, 2001), and/or and embryonic stem cells (Palacios et al., Proc. Nat. Acad. Sci. U.S.A., 92:7530-7534, 1995).

In some embodiments, the methods described herein include the use of a single dose of bone marrow. In the mouse models described herein, an allogeneic bone marrow dose of 25×10⁶ cells per recipient mouse is used. A living human donor can provide about 7.5×10⁸ bone marrow cells/kg. For human subjects, the methods described herein can include the administration of about 2.5×10⁸ cells/kg (e.g., for bone marrow), with higher doses used for peripheral blood stem cells. Sources of hematopoietic stem cells include bone marrow cells, mobilized peripheral blood cells, and cord blood cells. In some embodiments, mobilized peripheral stem cells are used. In vitro expanded hematopoietic cells can also be used.

In some embodiments, the stem cells are from a stem cell bank, or are from a donor identified using a database of stem cell donors, e.g., a donor identified as having a immune profile that matches a tissue or organ to be transplanted. In some embodiments, the stem cells are from the stem cell, tissue, or organ donor.

In some embodiments, the present methods include the use of an allogeneic bone marrow inoculum that is not T cell-depleted. It has been suggested that “facilitator” T cells may contribute to the establishment of allogeneic hematopoietic chimerism (Schuchert et al., Nat. Med., 6:904-909, 2000; Kaufman et al., Blood, 84:2436-2446, 1994; and Fowler et al., Blood, 91:4045-4050, 1998). The primary reason for T cell depletion of donor bone marrow in human transplantation is to reduce the risk of GVHD. In other embodiments, the present methods include the use of allogeneic bone marrow that has been T-cell depleted, e.g., using methods known in the art, such as anti-T cell depleting antibodies plus complement or anti-T cell antibody coated magnetic bead separation methods.

Tissue and/or Organ Transplantation

The methods describe herein have a number of clinical applications. For example, the methods can be used in a wide variety of tissue and organ transplant procedures, e.g., the methods can be used to induce tolerance in a recipient of a graft of stem cells such as bone marrow and/or of a tissue or organ such as pancreatic islets, liver, kidney, heart, lung, skin, muscle, neuronal tissue, stomach, and intestines. Thus, the new methods can be applied in treatments of diseases or conditions that entail stem cell tissue or organ transplantation (e.g., liver transplantation to treat liver failure, transplantation of muscle cells to treat muscular dystrophy, or transplantation of neuronal tissue to treat Huntington's disease or Parkinson's disease). In some embodiments, the methods include administering to a subject in need of treatment: 1) a T-cell specific siRNA delivery reagent complexed with an siRNA that specifically induces anergy and death of activated T cells; 2) a stem cell transplant, e.g., bone marrow, and 3) a donor organ or tissue, e.g., liver, kidney, heart, lung, skin, muscle, neuronal tissue, stomach and intestines.

As described herein, the tissue or organ will generally be from the same donor as the hematopoietic stem cell donor. In some embodiments, one individual will donate the hematopoietic stem cells and the tissue or organ. This will typically be the case where the donor is alive and viable, e.g., a volunteer donor of a regenerative or duplicated organ, e.g., a kidney, a portion of liver, or a bowel segment. In other embodiments, a first individual will donate the hematopoietic stem cells, and a second individual will donate the tissue or organ. This may more typically occur where the donors are, e.g., inbred animals, e.g., inbred pigs. In some embodiments, more than one individual will donate the stem cells, e.g., the population of stem cells will comprise cells from more than one donor.

In some embodiments, a donated tissue or organ is transplanted into the recipient once tolerance has been established, e.g., about two weeks, about four weeks, about six weeks, about eight weeks, about ten weeks or more after a stem cell transplant, i.e., a bone marrow transplant, as described herein. Typically, the tissue or organ transplant will take place four to eight weeks after the stem cell transplant. Evidence of central tolerance includes the establishment of hematopoietic chimerism, e.g., at least about 0.5%, 1.0%, 1.5%, 2%, 5%, 10%, 15%, or more of circulating peripheral blood mononuclear cells are of donor origin. Any suitable method can be used to evaluate the establishment of chimerism. As one example, flow cytometry can be used, e.g., using monoclonal antibodies to distinguish between donor class I major histocompatibility antigens and leukocyte common antigens versus recipient class I major histocompatibility antigens. Alternatively, chimerism can be evaluated by PCR. Tolerance to donor antigen can be evaluated by known methods, e.g., by mixed lymphocyte reaction (MLR) assays or cell-mediated lympholysis (CML) assays.

In some embodiments, a donated tissue or organ is transplanted in a recipient concurrently with a stem cell transplant, i.e., a bone marrow transplant, as described herein. In some embodiments, the recipient is then treated with a regimen of immune-suppressing drugs to prevent rejection of the tissue or organ, e.g., until hematopoietic chimerism and central tolerance are established. Minimal regimens of immunosuppressive treatment are known, and one of skill in the art would appreciate that the regimen should be selected such that the regimen should be such that engraftment of the bone marrow transplant should not be undermined Again, any suitable method can be used to evaluate the establishment of chimerism. Tolerance to donor antigen can be evaluated by known methods, e.g., by MLR assays or cell-mediated lympholysis (CML) assays.

In some embodiments, the donor is a living, viable human being, e.g., a volunteer donor, e.g., a relative of the recipient.

In some embodiments, the donor is no longer living, or is brain dead, e.g., has no brain stem activity. In some embodiments, the donor tissue or organ is cryopreserved.

In some embodiments, the donor is one or more non-human mammals, e.g., an inbred pig, or a non-human primate.

Other Applications

In addition to their use in tissue and organ transplants, the new methods can be used to treat a wide variety of disorders. For example, the new methods can be used to treat autoimmune diseases. Lymphohemopoietic cells with abnormal function have been implicated in this class of disorders, and these may be tolerized by induction of mixed chimerism using the methods described herein. The reversal of these autoimmune diseases by stem cell transplantation is likely to be associated with some degree of recovery in affected organ systems. For example, the present methods can be adapted to stem cell therapy protocols for the treatment of autoimmune disorders including, but not limited to, systemic lupus erythematosus, multiple sclerosis, rheumatoid arthritis, and scleroderma. A number of standard protocols are known, see, e.g., Sullivan and Furst, J. Rheumatol. Suppl., 48:1-4, 1997; Burt and Traynor, Curr. Op. Hematol., 5:472-7, 1998; Burt et al., Blood, 92(10):3505-14, 1998; Openshaw et al., Biol. Blood Marrow Transplant., 8:233-248, 2002. Accordingly, the invention includes methods for treating an autoimmune disorder, by administering to a subject in need of treatment: 1) a T-cell specific siRNA delivery reagent complexed with an siRNA that specifically induces anergy and death of activated T cells; and 2) a stem cell transplant, e.g., bone marrow.

One of skill in the art will appreciate that the methods described herein can be adapted for the treatment of malignancy, e.g., hematological malignant disease. Immunocompetent donor cells, transplanted with the stem cells, have potent graft-versus-tumor activity (GVT) (see, e.g., Appelbaum, Nature, 411:385-389, 2001). The new methods provide (1) durable, sustained engraftment of stem cells without inducing GVHD, and (2) donor-antigen specific transplant tolerance. This allows administration of non-tolerant donor lymphocytes to mediate GVT effects. This can occur without GVHD under these conditions. Thus, the new methods separate the GVT activity and GVHD activity, allowing the GVT response to be strengthened while avoiding GVHD, and are safer and far less toxic than conventional methods. Thus, the present invention includes methods of treating a subject having a hematologic malignancy, e.g., leukemia, by administering to the subject 1) a T-cell specific siRNA delivery reagent complexed with an siRNA that specifically induces anergy and death of activated T cells; and 2) a stem cell transplant, e.g., bone marrow, under conditions suitable for the donor stem cells to exert a graft-versus-tumor effect.

The new methods can also be used to treat genetic disorders, e.g., hematologic disorders cause by a genetic mutation, such as beta-thalassemia and sickle cell. See, e.g., Yang and Hill, Pediatr. Infect. Dis. J., 20:889-900, 2001; and Persons and Nienhuis, Curr. Hematol. Rep., 2(4):348-55, 2003. Thus, the invention also includes methods for the treatment of a genetic disorder in a subject, by administering to the subject 1) a T-cell specific siRNA delivery reagent complexed with an siRNA that specifically induces anergy and death of activated T cells; and 2) a stem cell transplant, e.g., bone marrow cells. In some embodiments, the cells of the stem cell transplant can be genetically modified, e.g., to express a particular protein that is useful in treating the genetic disorder. In some embodiments, the stem cells are from a donor who does not have the genetic disorder (e.g., normal stem cells), and the presence of the normal stem cells is sufficient to treat the genetic disorder.

The new methods can also be used to facilitate gene therapy (Bordignon and Roncarolo, Nat. Immunol., 3:318-321, 2002; Emery et al., Int. J. Hematol., 75:228-236, 2002; Park et al., Gene Ther., 9:613-624, 2002; Desnick and Astrin, Br. J. Haematol., 117:779-795, 2002; Bielorai et al., Isr. Med. Assoc. J., 4:648-652, 2002). Thus, in some embodiments, the stem cells are genetically altered, e.g., have at least one genetic modification, e.g., a modification that alters the expression of at least one gene, e.g., alters the level, timing, or localization of at least one gene.

In some embodiments, other treatments can be administered in combination with siRNAs, including but not limited to partial T cell depletion (e.g., using low-dose injections of depleting anti-CD4 and anti-CD8a mAbs, or PD-L1.Ig and anti-CD25 to deplete activated T cells) prior to BMT. Additionally, studies in the costimulation blockade-based model have indicated that use of either a blocking anti-OX40L antibody or a CTLA4Ig fusion protein improves tolerization and, therefore, might be beneficial and non-toxic in combination with siRNA delivery. In some embodiments, the methods include administration of anti-CD154 antibodies, or administration of low-dose radiation.

### EXAMPLES

The invention is further described in the following examples, which do not limit the scope of the invention described in the claims.

### Example 1

### Construction of ICAM-1 D1D2 Fusion Proteins

As a method of delivering siRNAs into activated, donor-reactive T cells, the ICAM-1-LFA-1 interaction was exploited. A fusion protein containing a portion of mouse ICAM-1 that confers HA LFA-1 specificity, namely domain 1 (D1) and domain 2 (D2), was constructed to permit delivery of siRNAs only to HA LFA-1-expressing cells and not to cells expressing Mac-1 or other ICAM-1 ligands. The ICAM-1 region used is predicted to bind both the human and mouse proteins (11;84). The conserved Kozak sequence (GCCACCAUGG; SEQ ID NO:5) for ribosome binding and translation initiation was fused to D1 and D2 of murine ICAM-1, which was subsequently fused with a portion of human IgG Fc (C_{H}2 and C_{H}3), a flexible linker (GGGS). This sequence is fused to a cationic sequence of amino acids 8 through 29 of His6-tagged human protamine, enabling electrostatic binding of negatively charged siRNA molecules (FIG. 1). A secretion signal peptide (sequence: MASTRAKPTLPLLLALVTVVIPG (SEQ ID NO:1)) in exon 1 of ICAM-1 was included to permit secretion of the fusion protein. The Fc region was included to facilitate ICAM-1 dimerization, which increases avidity for HA LFA-1, and to enable pull-down of the fusion protein using protein A agarose beads.

The nucleic acid sequence of the ICAM-protamine construct was:

(SEQ ID NO: 6)
ATGgcttcaacccgtgccaagcccacgctacctctgctcctggccctggtcaccgttgtgatc
cctgggcctggtgatgctcaggtatccatccatcccagagaagccttcctgccccagggtggg
tccgtgcaggtgaactgttcttcctcatgcaaggaggacctcagcctgggcttggagactcag
tggctgaaagatgagctcgagagtggacccaactggaagctgtttgagctgagcgagatcggg
gaggacagcagtccgctgtgctttgagaactgtggcaccgtgcagtcgtccgcttccgctacc
atcaccgtgtattcgtttccggagagtgtggagctgagacctctgccagcctggcagcaagta
ggcaaggacctcaccctgcgctgccacgtggatggtggagcaccgcggacccagctctcagca
gtgctgctccgtggggaggagatactgagccgccagccagtgggtgggcaccccaaggacccc
aaggagatcacattcacggtgctggctagcagaggggaccacggagccaatttctcatgccgc
acagaactggatctcaggccgcaagggctggcattgttctctaatgtctccgaggccaggagc
ctccggactttcgcgGgatccGACAAAACTCACACATGCCCACCGTGCCCAGCACCTGAACTC
gcGGGGGcACCGTCAGTCTTCCTCTTCCCCCCAAAACCCAAGGACACCCTCATGATCTCCCGG
ACCCCTGAGGTCACATGCGTGGTGGTGGACGTGAGCCACGAAGACCCTGAGGTCAAGTTCAAC
TGGTACGTGGACGGCGTGGAGGTGCATAATGCCAAGACAAAGCCGCGGGAGGAGCAGTACAAC
AGCACGTACCGGGTGGTCAGCGTCCTCACCGTCCTGCACCAGGACTGGCTGAATGGCAAGGAG
TACAAGTGCAAGGTCTCCAACAAAGCCCTCCCAGCCCCCATCGAGAAAACCATCTCCAAAGCC
AAAGGGCAGCCCCGAGAACCACAGGTGTACACCCTGCCCCCATCCCGGGATGAGCTGACCAAG
AACCAGGTCAGCCTGACCTGCCTGGTCAAAGGCTTCTATCCCAGCGACATCGCCGTGGAGTGG
GAGAGCAATGGGCAGCCGGAGAACAACTACAAGACCACGCCTCCCGTGCTGGACTCCGACGGC
TCCTTCTTCCTCTACAGCAAGCTCACCGTGGACAAGAGCAGGTGGCAGCAGGGGAACGTCTTC
TCATGCTCCGTGATGCATGAGGCTCTGCACAACCACTACACGCAGAAGAGCCTCTCCCTGTCT
CCGGGTAAAGGTGGAGGCGGTTCAGCGGCCGCACGCAGCCAGAGCCGGAGCAGATATTACCGC
CAGAGACAAAGAAGTCGCAGACGAAGGAGGCGGAGCCTCGAGCACCACCACCACCACCACtga
g

The amino acid sequence of the ICAM-protamine fusion protein was:

(SEQ ID NO: 7)
MASTRAKPTLPLLLALVTVVIPGPGDAQVSIHPREAFLPQGGSVQVNCSSSCKEDLSLGLETQ
WLKDELESGPNWKLFELSEIGEDSSPLCFENCGTVQSSASATITVYSFPESVELRPLPAWQQV
GKDLTLRCHVDGGAPRTQLSAVLLRGEEILSRQPVGGHPKDPKEITFTVLASRGDHGANFSCR
TELDLRPQGLALFSNVSEARSLRTFAGSDKTHTCPPCPAPELAGAPSVFLFPPKPKDTLMISR
TPEVTCVVVDVSHEDPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMHEALHNHYTQKSLSLS
PGKGGGGSAAARSQSRSRYYRQRQRSRRRRRRSLEHHHHHH

The fused sequence was ligated into the pcDNA 3.1 mammalian expression vector (from Invitrogen) prior to transfecting the plasmid (using LIPOFECTAMINE™ 2000 lipofection reagent from Invitrogen) into Chinese Hamster Ovary (CHO) Lec 3.2.8.1 cells. This CHO variant is a mammalian cell line with 4 glycosylation mutations that result in truncated N- and O-linked carbohydrates while maintaining efficient cell growth rates. In the CHO Lec 3.2.8.1 cells, N-linked sugars are in the Man5 oligomannosyl form, and O-linked sugars are truncated to a single N-acetyl galactosamine (Ga1NAc) (85). These truncations facilitate production of glycoproteins that are biologically active with minimal carbohydrate heterogeneity. Use of this cell line is ideal for expression of the ICAM-1 construct because the proteins produced by them are homogeneous in their glycosylation and do not bind to most lectins, which protects from binding to untargeted cell types.

Although Nickel-affinity chromatography could be used to purify the protein even further by utilizing the His tag, pull-down from the medium using protein A agarose beads has been efficient at purifying the ICAM-1 fusion protein. This is shown in FIG. 2 by Western blotting (WB) using both an anti-murine ICAM-1 antibody (binding within D1D2) and an anti-human protamine antibody (duplicate wells, binding within aa 8-29). A clean band at the expected 54 kD size was observed after concentration of medium from ICAM-1 construct-expressing CHO Lec 3.2.8.1 cells and pull-down (PD) with protein A agarose beads.

### Example 2

### Cell Binding Specificity Assays

Preliminary cell adhesion assay results suggest that the ICAM-1 fusion protein binds preferentially to HA LFA-1 (murine and human). V-bottom wells were coated with 10, 5, or 1 μg/mL of the ICAM-1 construct. As depicted in FIG. 3, cells expressing HA LFA-1 (TK1 +Mn and K562 HA LFA-1) show a greater percent adhesion than cells expressing WT LFA-1 (TK1 −Mn and K562 WT LFA-1) or no LFA-1 (K562).

The assay was performed essentially as previously described (76;86). The concept of this assay is that activated, labeled cells added to V-bottom wells coated with purified fusion protein will bind to the fusion protein and, therefore, will not pellet when centrifuged at a slow speed. Unactivated cells, by contrast, will pellet to the bottom of the well because the cells will not adhere to the immobilized fusion protein (76;86). This assay avoids a washing step that could dislodge activated, bound cells from the wells. Polypropylene, V-bottom, 96-well plates were coated overnight at 37° C. with various concentrations of the ICAM-1 construct starting at 10 μg/mL and titrated down. Control wells were coated with full-length ICAM-1 as a positive control and BSA or protamine alone as negative controls. A standard carbonate bicarbonate basic coating buffer was used. As an additional control for specificity, a second set of titrated ICAM-1 construct-coated wells were made and blocked by addition of excess anti-ICAM-1 mAb, YN 1/1.7, to show inhibition of binding of activated cells (87). After washing the wells with PBS, a blocking buffer consisting of PBS with 2% BSA will be added and incubated at 37° C. for 1 hour. After washing, 50 μL of media with or without 2 mM MnCl₂ was added to the wells and warmed to 37° C.

TK1 cells, which are a murine T cell lymphoma cell line (CD8+ and CD4+CD8+), were incubated with 2′,7′-bis-(carboxyethyl)-5-(and -6)-carboxyfluorescein acetoxymethyl ester (BCECF-AM) for 15 minutes at 37° C. for labeling, washed, and 3×10⁴ cells in 50 μL were added to the coated wells containing medium. The final concentration of MnCl₂ in the “activating” wells was 1 mM, which reliably converts LFA-1 to its high affinity conformation. Immediately upon addition of the cells to the coated wells, the V-bottom plate was centrifuged at 700 rpm for 15 minutes to promote interactions between immobilized ICAM-1 construct and the cells. The plate was then read using a fluorescence plate reader with a 485 nm excitation filter and a 535 nm emission filter to determine the fluorescence at the center of the V-bottom wells. Since adherent cells did not pellet, less fluorescence indicated more binding. The percent adhesion was calculated according to the formula: % adhesion=100−(sample mean/background mean×100), where sample mean is the mean fluorescence of the experimental well and background mean is the mean fluorescence of the well coated with BSA. The results of these assays demonstrate specificity of binding.

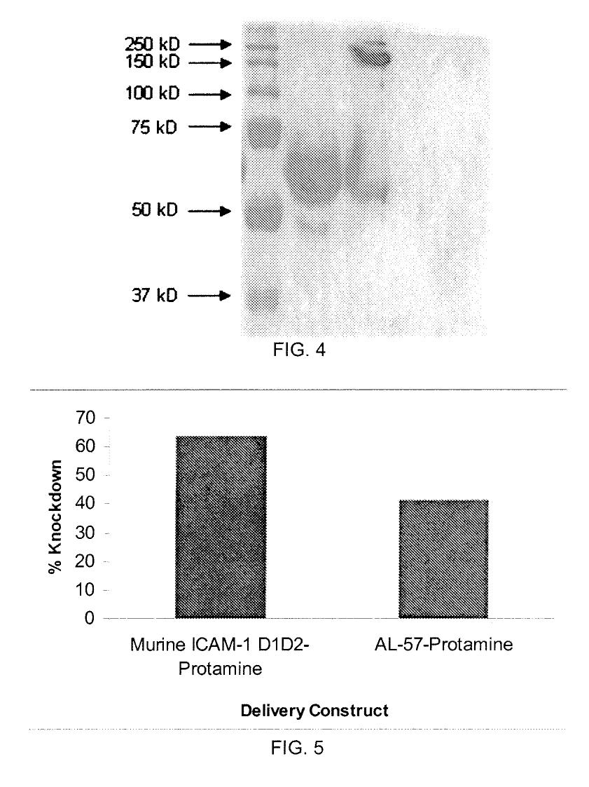

The cross-reactivity of the ICAM-1-protamine fusion protein for human HA LFA-1 will be highly advantageous for translating encouraging mouse results into clinical proof-of-concept studies. As shown in FIG. 4, the ICAM-1-protamine fusion protein dimerized as expected via interactions between the Fc fragments. This construct was used with 10 μmol Ku70 siRNA (a 6:1 ratio of siRNA to construct). As shown in FIG. 5, the murine ICAM-1-Fc-protamine construct was slightly more effective in knocking down Ku70 expression in HA LFA-1-transfected K562 cells than the AL-57-protamine anti-HA LFA-1 mAb. These studies demonstrate the use of the murine ICAM-1 construct to target siRNAs to HA-LFA-1-expressing cells.

In addition to the V-bottom adherence assay described above, a flow cytometry-based binding assay was developed to assess binding in a more physiologically relevant system and in the hope of achieving more specific binding with less background in unactivated cells. In this assay, various ratios of ICAM-1-protamine fusion protein to polyclonal goat anti-human Fc Fab′2 were used to optimize conditions for binding to primary murine splenocytes. The readout was fluorescence intensity of APC conjugated to the anti-Fc Fab′2 fragments. The fusion protein and anti-Fc were incubated in a volume of 60 uL for 30 min at 4° C. before the complexes were added to 1×10⁶ primary murine splenocytes that were either unactivated (PBS) or artificially activated (PBS containing 20 mM Hepes, 5 mM MgCl2, and 1 mM EGTA). The total volume during the binding reaction was 100 uL to promote optimal contact between multimers and cells. After incubating the labeled multimers with cells for 20-30 min at room temperature, the cells were fixed with 500 uL of warm 4% paraformaldehyde for 15-30 min then washed and analyzed. For the first experiment, the amount of anti-Fc Fab′2 was held constant at 10 pmoles (which corresponds to 1 ug of anti-Fc Fab′2) and the amount of ICAM-1-protamine fusion protein was varied.

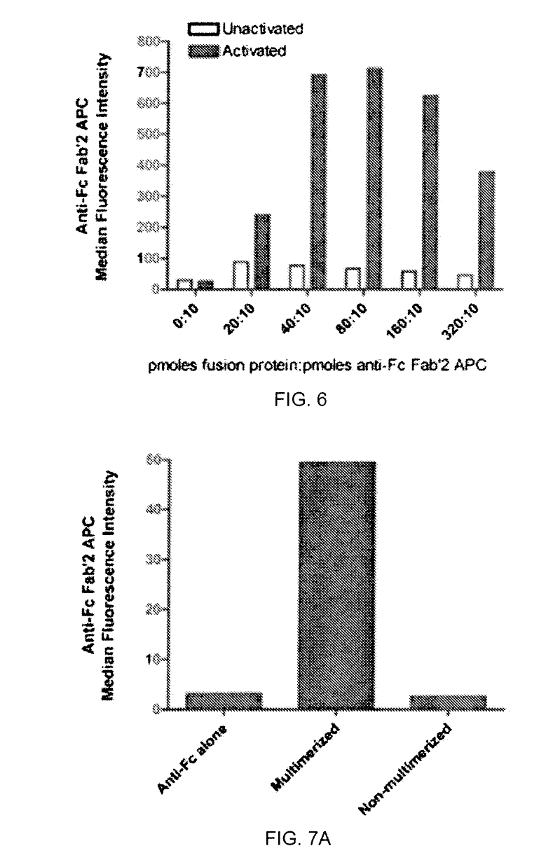

The highest binding to activated cells with minimal binding to unactivated cells was obtained when 80 pmoles of ICAM-1-protamine fusion protein were complexed with 10 pmoles of anti-Fc Fab′2 (FIG. 6). These results confirm the preliminary data using the V-bottom assay and show specific binding to activated leukocytes. Moreover, the results demonstrate that optimal complex formation occurs when fusion protein and anti-Fc are mixed at an 8:1 ratio. Higher ratios led to reduced binding, likely because the excess of fusion protein soaks up the anti-Fc Fab′2 and prevents formation of multimers with sufficient valency of ICAM-1. This experiment was repeated and confirmed that optimal binding was observed when 80 pmoles fusion protein and 10 pmoles anti-Fc Fab′2 were used, e.g., an 8:1 ratio.

### Example 3

### Multimerization Increases Binding to Activated Cells

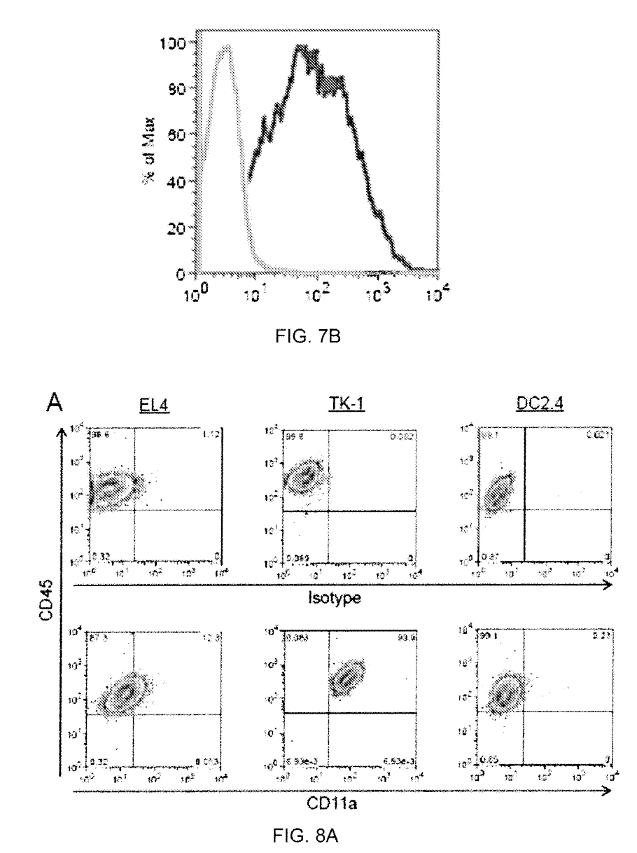

A flow cytometry-based binding assay was used to determine if the ICAM-1-protamine fusion protein could bind activated primary murine splenocytes without first being multimerized. For the multimer control, 80 pmoles of ICAM-1-protamine fusion protein and 10 pmoles of anti-Fc Fab′2 APC were pre-incubated before being added to artificially activated cells. For the non-multimerized sample, artificially activated cells were first incubated with 80 pmoles of fusion protein then fixed, washed, and incubated with anti-Fc Fab′2 APC as a secondary stain. Importantly, no binding was observed in the non-multimerized condition (FIGS. 7A-B). As such, all subsequent knockdown experiments were performed using ICAM-1-protamine fusion protein that was multimerized with anti-Fc Fab′2.

### Example 4

### In Vitro Knockdown

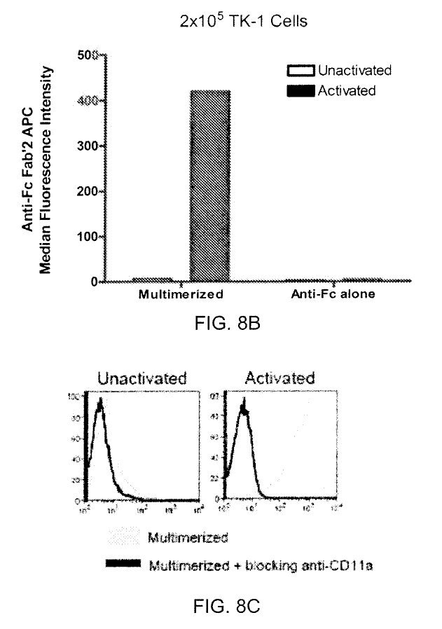

Initial in vitro knockdown studies were performed using non-multimerized ICAM-1-protamine fusion protein and were unsuccessful since, as described in Example 3, efficient binding to HA LFA-1 requires multimerization. To optimize delivery of siRNAs using ICAM-1-protamine fusion protein multimers in vitro, initial knockdown experiments were designed in cell lines. Flow cytometry was first used on several hematopoietic cell lines to confirm robust expression of CD11a (the alpha chain of LFA-1) and CD45 (the target for our validation siRNA). Expression was examined in EL4, TK-1, and DC2.4 cell lines, which are murine T cell, thymocyte, and dendritic cell lines, respectively (FIG. 8A). All cells expressed high levels of CD45; however, only TK-1 cells expressed adequate levels of CD11a.

Before proceeding with in vitro knockdown studies, it was first confirmed that functional LFA-1 was expressed on TK-1 cells by performing the flow cytometry-based multimer binding assay on these cells. These studies demonstrated that the multimerized fusion protein (80 pmoles fusion protein with 10 pmoles anti-Fc Fab′2) bound well to 2×105 (and also 4×105, not shown) artificially (Mg and EGTA) stimulated TK-1 cells (FIG. 8B). Moreover, this binding is specific for LFA-1 (and not Mac-1 or another ICAM-1 ligand), as it is completely inhibited when the TK-1 cells are first incubated with a blocking anti-CD11a mAb to prevent binding of ICAM-1 in the fusion protein multimer to LFA-1 on the cells (FIG. 8C).

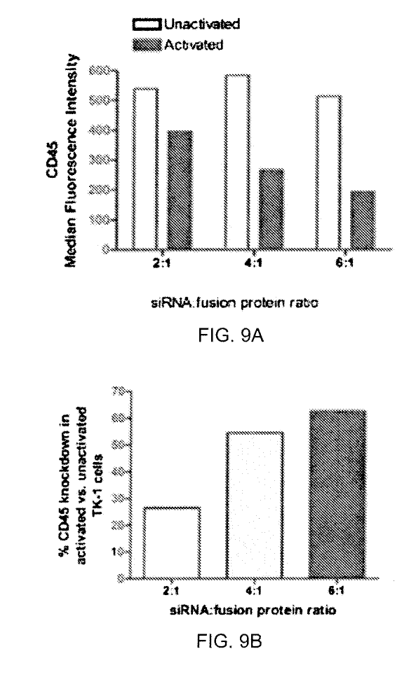

Next, CD45 siRNA was delivered into activated (Mg and EGTA) or unactivated (PBS) TK-1 cells in vitro. For the initial experiment, 2×10⁵ TK-1 cells were used, the amount of ICAM-1-protamine fusion protein was held constant at 80 pmoles, and the amount of anti-Fc Fab′2 was held constant at 10 pmoles. The siRNA amount was varied to make the molar ratio of siRNA:fusion protein 2:1, 4:1 or 6:1. These conditions were chosen since the multimer binding assay showed that this amount of fusion protein and anti-Fc gave robust binding results even with 4×105 cells, indicating that it would be an excess when 2×10⁵ cells are used. First, the siRNA and fusion protein were incubated in a volume of 40 uL of medium for 30 mins at room temperature. Next, the anti-Fc Fab′2 was added to bring the multimer volume to 60 uL and incubated for 30 mins at 4° C. The 60 uL of complexes were dripped onto activated or unactivated TK-1 cells in 40 uL of medium in a 96-well plate. Five hours later, the cells were spun and washed, and the medium was replaced with 200 uL fresh medium containing 5% FCS. The cells were incubated for 72 hours post-transfection, then harvested and analyzed for surface CD45 expression. There was an incremental decrease in CD45 expression in the major population when comparing the 2:1, 4:1, and 6:1 ratios of siRNA to fusion protein. Interestingly, at the 6:1 ratio, a distinct subpopulation of CD45-low cells that constituted 14% of the total population was seen (FIGS. 9A-B).

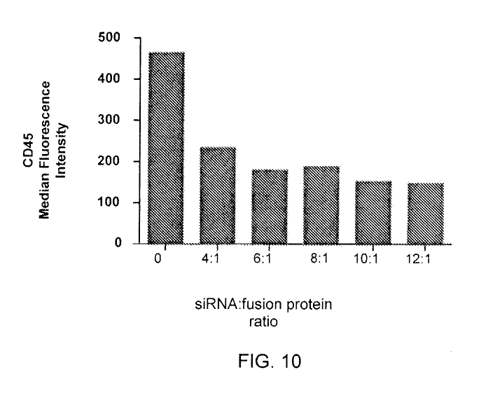

Furthermore, increasing the ratio of siRNA to fusion protein and using a lower concentration 2-3% fetal calf serum (FCS) allows enhanced knockdown of CD45 expression in all cells (FIG. 10). In this experiment, no subpopulation of CD45-negative cells was observed. This is shown in a murine thymoma cell line, which is significant since mouse T cells are notoriously difficult to transfect.

These data demonstrate that robust knockdown can be achieved specifically in activated cells in vitro.

### Example 5

### Gene Silencing in In Vivo-Activated T cells

In vitro gene silencing is assessed in mouse T cells activated in vivo by exposure to allogeneic BMCs. To generate mice for this purpose, C57BL/6 (B6) mice receive 3 Gy TBI followed 6 hours later by i.v. injection of 5×10⁶ syngeneic 2C TCR Tg and 5×10⁶ syngeneic 4C TCR Tg (on a Rag knockout background) BMCs (66;67). These 2C.4C.B6 synchimeric mice will reconstitute their hematopoietic system with a small population of CD8 T cells (2C) and CD4 T cells (4C) that bear a transgenic TCR specific for MHC class I and class II molecules of the H-2d haplotype, respectively. After allowing 6 weeks for reconstitution, the percentage of CD8 T cells that bear the 2C TCR and the percentage of CD4 T cells that bear the 4C TCR are evaluated in the peripheral blood. To do this, the clonotypic 1B2 antibody is used to identify 2C+CD8 T cells, and, since no clonotypic antibody is available for the 4C TCR, anti-Vβ13 and anti-Thy1.1 antibodies are used to identify 4C+CD4 T cells, which express the Thy1.1 congenic marker. Mice with 5-20% of their CD8 and CD4 T cells bearing the 2C and 4C receptor, respectively, are then given an allogeneic or syngeneic BMT. Robust activation of donor-reactive T cells occurs in recipients of allogeneic BMCs, which will be rejected. B10.D2 mice have the H-2d MHC genotype and will therefore be recognized by the TCR Tg cells in 2C.4C.B6 synchimeras. Neither the MHC class I nor class II antigens recognized by 2C and 4C cells are expressed by B10.S mice, which are used as irrelevant allogeneic donors. A third group receives syngeneic B6 BMT. Exposure to cognate allogeneic MHC molecules in recipients of B10.D2 BMT will activate 2C and 4C cells in vivo, resulting in conversion of LFA-1 to its HA conformation through inside-out signaling in these traceable donor-specific T cells.

First, the kinetics of conversion of LFA-1 to its HA conformation are examined by sacrificing 2C.4C.B6 synchimeras at various times after administration of allogeneic or syngeneic BMCs. Their spleens are harvested and processed for flow cytometric analysis of HA LFA-1 expression using labeled ICAM-1 Fc. Staining with ICAM-Fc is compared with staining with a conformation-independent anti-LFA-1 mAb. Once the kinetics of LFA-1 upregulation and conversion to the HA conformation are established, 2C.4C.B6 synchimeras receive relevant or irrelevant allogeneic or syngeneic marrow and sacrificed at various times. Polyclonal and 2C and 4C T cells are sorted, then incubated with the ICAM-1 construct complexed with the validated siRNA against mouse CD45. Knockdown of CD45 is evaluated using qRT-PCR and flow cytometry. Thereby the level of knockdown obtained with a validated siRNA molecule delivered with the ICAM-1 fusion protein into cells activated or not in vivo by allogeneic BMCs is assessed.

### Example 6

### In Vivo siRNA Delivery

The function of the protamine-containing ICAM-1 construct in vivo is characterized. Cell type-specific delivery of siRNAs in vivo has been successfully achieved by incubating 6 nmol of total siRNA with the delivery fusion protein at a 4:1, 6:1, 8:1, 10:1, or 12:1 ratio in PBS for 30 minutes at room temperature and injecting the complex i.v. in a volume of 100 μL into mice (11;12). In vivo delivery and knockdown is studied using the K562 mouse lung tumor model previously described (11). Delivery of fluorescent siRNAs into K562 cells expressing either WT or HA human LFA-1 that have formed lung tumors in immunodeficient mice is evaluated by flow cytometry and fluorescence microscopy after i.v. injection of siRNA-fusion protein complexes, as described. The TS1/22-protamine (conformation insensitive) and AL57-protamine (specific for HA LFA-1) fusion proteins are used as positive controls for these experiments. Knockdown of human Ku70 in these tumors is evaluated by immunohistochemistry and qRT-PCR analysis.

Specificity of delivery is evaluated in the BMT model using 2C.4C.B6 mice with 5-20% of their CD8 and CD4 T cells bearing the 2C and 4C receptor, respectively. These animals are given allogeneic or syngeneic BMT (25×10⁶) with no conditioning other than 3 Gy TBI on Day-1. Without the addition of anti-CD154 mAb, these mice uniformly reject bone marrow allografts. At the time of BMT, fluorescently labeled siRNAs complexed with the ICAM-1 construct is injected i.v. An additional injection of siRNAs is given the day after BMT and possibly at additional time points, before and/or after the BMT, depending on the kinetics of HA-LFA1 expression. Four hours after the final siRNA:construct injection, the spleens of mice that receive either relevant (B10.D2) or irrelevant (B10.S) or syngeneic (B6) BMCs are harvested, and cell suspensions will be prepared for flow cytometric analysis. The cell suspensions are stained with 1B2, anti-CD8β, anti-Vβ13, anti-Thy1.1, and anti-CD4 to look for colocalization of the fluorescently labeled siRNA that was injected in vivo. The percentages of 2C and 4C cells that have taken up the labeled siRNA are determined. Non-specific delivery is evaluated by examining the uptake of labeled siRNAs by 2C and 4C TCR Tg cells in mice receiving irrelevant B10.S and syngeneic B6 BMT. Several different quantities of siRNA are used with different dosing schedules to find the optimal conditions for efficient delivery while maintaining reasonable (and clinically feasible) doses. These studies provide information about the specificity and efficiency of delivery in vivo.

Given that the injected construct will have to out-compete binding of endogenous ICAM-1 to HA LFA-1, it is expected that a larger quantity of construct will need to be injected than previously reported, since the prior systems involved introduced cells engineered to express a unique antigen targeted by the delivery construct. Therefore, the starting amount is 6 nmol of siRNA and 1 nmol of construct and is titrated up to determine the range of efficacy for this reagent.

Next, the efficacy and kinetics of knockdown in vivo are investigated. A similar procedure to that described above for determining specificity and efficiency of delivery of fluorescently labeled siRNAs is used to determine the level and kinetics of knockdown. However, for these studies 2C.4C.B6 synchimeras receive validated siRNAs silencing CD45 or a scrambled control siRNA. The complexes are delivered i.v. at the time of allogeneic B10.D2 or B10.S or syngeneic B6 BM injection. Again, the quantity, number, and timing of injections will be varied based on the in vivo results obtained in the studies described above. RNA from FACS sorted 2C and 4C cells from the spleen and lymph nodes are extracted at different timepoints in order to evaluate the presence and level of siRNA and CD45 mRNA within the cells using modified Northern blotting. Additionally, knockdown of CD45 protein is examined using flow cytometry. These results are compared in cells taken from synchimeras receiving B10.D2 versus B10.S or B6 BMCs.

### Example 7

### Specific Silencing of Lethality Genes in Activated T Cells is Sufficient to Anergize and Delete Donor-Reactive Cells

The following experiments are performed to confirm that delivery of siRNAs silencing lethality genes, e.g., RasGRP1, cyclin D1, and bcl-xL, directly into activated donor-reactive T cells can promote induction of mixed chimerism.

The siRNA sequences used in vivo are obtained from commercial sources (e.g., Dharmacon) or determined in silico using siRNA design tools, which are available from many sources (e.g., the selection program described in Yuan et al., Nucl. Acids. Res. 32:W130-W134 (2004), available online at jura.wi.mit.edu/siRNAext/home.php, which utilizes a collection of rules that have empirically been shown to predict the most effective siRNA molecules, originally disclosed in Elbashir et al., Genes Dev. 15(2):188-200 (2001); Schwarz et al., Cell. 115(2):199-208 (2003); Khvorova et al., Cell. 115(2):209-16 (2003); Pei and Tuschl, Nat. Methods. 3(9):670-6 (2006); Reynolds et al., Nat. Biotechnol. 22(3):326-30 (2004); Hsieh et al., Nucleic Acids Res. 32(3):893-901 (2004); and Ui-Tei et al., Nucleic Acids Res. 32(3):936-48 (2004)).

Each siRNA is validated by transfecting into TK1 cells or, if necessary, more readily transfected murine cells such as NIH3T3 fibroblasts or P815 murine mastocytoma cells that have previously been transfected with the target gene (i.e., RasGRP1, cyclin D1, or bcl-xL). An siRNA is considered valid if it demonstrates 70% or more knockdown of the target transcript with no detectable knockdown of the top three transcripts (determined by BLAST homology) with the most similar sequence. If inclusion of more than one siRNA targeting the same transcript is found to result in significantly improved knockdown, the cocktail is used for in vivo studies. As a stringent control for specificity, the murine cell line is transfected with expression vectors containing an unmutated or a mutated (with conserved amino acid sequence but altered DNA and mRNA sequence) sequence encoding the transcript that is being targeted by the siRNA under investigation. Upon transfection with the siRNA molecule, only the cells expressing the unmutated gene are expected to demonstrate diminished mRNA and protein levels relative to cells that don't receive the siRNA. The silent mutation should prevent recognition by the siRNA sequence and, therefore, will serve as a stringent test for off-target effects. Additionally, the siRNAs have modifications that will promote stability and effective knockdown in vivo. These modifications may include a phosphorothioate (P═S) backbone linkage at the 3′ end, a 2′-O-methyl uridine or guanosine, and a mismatch at the 5′ end of the active strand.

To demonstrate that silencing of RasGRP1, cyclin D1, and bcl-xL transcripts is sufficient to delete activated T cells, 2C and 4C cells taken at various times (optimized in Aim 1) from synchimeras receiving B10.D2 (relevant) or B10.S (irrelevant) or B6 (syngeneic) BMT are sorted and subjected to delivery of these siRNA molecules using the ICAM-1 construct ex vivo. Upon demonstrating that ex vivo delivery results in anergy (by MLR and CML) and death of in vivo-activated T cells, the 2C.4C.B6 synchimeras will be used to demonstrate efficacy in vivo. The level of 2C and 4C cells in the peripheral blood is evaluated prior to and at several timepoints after BMT with B10.D2, B10.S, or B6 BMCs when siRNAs silencing RasGRP1, cyclin D1, and bcl-xL are delivered i.v. using the ICAM-1 construct. If, as expected, silencing of these transcripts is sufficient to prevent expansion and induce apoptosis of activated leukocytes, the activated donor-specific Tg T cells should be rapidly deleted. If this is not observed in the ex vivo and in vivo studies, other siRNAs are tested to improve deletion. Other potential siRNA targets include bcl-2, mcl-1, Akt, N-ras, SOS, Zap70, mTOR, NFAT, NFkB, HSP90, polo-like kinases (plk), cFLIP, ICAD, and/or several other proteins involved in T cell activation and survival, e.g., survivin. Additionally, a combination of different constructs targeting different activation-induced cell surface antigens may be used for delivery of the cocktail of siRNAs. For example, the ICAM-1 construct in combination with constructs (made using scFvs fused to protamine, for instance) targeting CD69 are injected simultaneously to enhance delivery to activated, alloreactive T cells.

Based on the in vivo kinetics data, the dosing amount and schedule is optimized. To perform in vivo tolerance experiments, the standard costimulation blockade-based regimen is modified such that the anti-CD154 injection is replaced by injections of siRNA:construct complexes. Female 2C.4C.B6 mice receive 3 Gy TBI on Day-1 followed by 25×10⁶−40×10⁶ (depending on the level of 2C and 4C donor-reactive cells in the periphery) female B10.D2, B10.S or B6 BMCs on Day 0. The 2C.4C.B6 recipients in the positive control allogeneic BMT groups receive 2 mg of anti-CD154 (MR1) i.p. on Day 0 (our established costimulation blockade-based regimen). The 2C.4C.B6 recipients in the negative control allogeneic BMT groups receive i.v. injection of siRNA silencing eGFP (which is not expressed in these mice) complexed to the ICAM-1 construct. The antisense strand of the siRNA molecule for eGFP have the following sequence: 5′-AAGCAGCAGGACUUCUUCAAG-3′ (106; SEQ ID NO:XX). The 2C.4C.B6 recipients in the experimental groups receive complexes containing a cocktail of siRNAs silencing RasGRP1, cyclin D1, and bcl-xL with the ICAM-1 construct. The siRNA complex injection dose and schedule is determined empirically, starting with 80 mg of total siRNA at a ratio of 6:1 with the ICAM-1 construct injected 5 hours after BMC injection and subsequently on Days 1, 3, 5, 8 and 10. Additional control groups receive syngeneic marrow with the ICAM-1 complexed to the RasGRP1/cyclin D1/bcl-xL siRNA cocktail or to the eGFP siRNA. The recipients in all groups are followed long-term to evaluate deletion of 2C+CD8 and 4C+CD4 T cells, total CD4 and CD8 T cell counts, and the progression of mixed chimerism in the peripheral blood using flow cytometry to detect donor MHC class I (using anti-H2 Dd mAb 34-2-12) on B cells, myeloid cells, CD4 T cells, and CD8 T cells over time. Tolerance is evaluated by donor and third party skin grafting 50 days post-BMT, as well as CML and MLR assays and measurements of deletion of thymic and peripheral 2C and 4C cells at the time of euthanasia 6 months post-BMT.

In some experiments, siRNA injections are begun 5 hours after donor BMC injection because LFA-1 is involved in, though not crucial for, homing of circulating HSCs to the bone marrow (107-111). This homing process is very rapid and, therefore, should not be hindered if the ICAM-1 construct is delivered 5 hours after BMC injection (112). If no chimerism is achieved despite demonstrable deletion in the validation experiments, the dosing and injection schedule is modified to ensure HSC homing is unperturbed, e.g., by injecting the siRNA complexes later to give HSCs time to home.

### REFERENCES

1. Sharabi, Y. and Sachs, D. H. 1989. J Exp Med 169:493-502.
2. Sharabi, Y., Abraham, V. S., Sykes, M., and Sachs, D. H. 1992. Bone Marrow Transplant 9:191-197.
3. Tomita, Y., Sachs, D. H., and Sykes, M. 1994. Blood 83:939-948.
4. Wekerle, T., Sayegh, M. H., Hill, J., Zhao, Y., Chandraker, A., Swenson, K. G., Zhao, G., and Sykes, M. 1998. J Exp Med 187:2037-2044.
5. Takeuchi, Y., Ito, H., Kurtz, J., Wekerle, T., Ho, L., and Sykes, M. 2004. Am J Transplant 4:31-40.
6. Ferrant, J. L., Benjamin, C. D., Cutler, A. H., Kalled, S. L., Hsu, Y. M., Garber, E. A., Hess, D. M., Shapiro, R. I., Kenyon, N. S., Harlan, D. M. et al. 2004. Int Immunol 16:1583-1594.
7. Kanmaz, T., Fechner, J. J., Jr., Torrealba, J., Kim, H. T., Dong, Y., Oberley, T. D., Schultz, J. M., Bloom, D. D., Katayama, M., Dar, W. et al. 2004. Transplantation 77:914-920.
8. Kawai, T., Andrews, D., Colvin, R. B., Sachs, D. H., and Cosimi, A. B. 2000. Nat Med 6:114.
9. Koyama, I., Kawai, T., Andrews, D., Boskovic, S., Nadazdin, O., Wee, S. L., Sogawa, H., Wu, D. L., Smith, R. N., Colvin, R. B. et al. 2004. Transplantation 77:460-462.
10. Schuler, W., Bigaud, M., Brinkmann, V., Di Padova, F., Geisse, S., Gram, H., Hungerford, V., Kleuser, B., Kristofic, C., Menninger, K. et al. 2004. Transplantation 77:717-726.
11. Peer, D., Zhu, P., Carman, C. V., Lieberman, J., and Shimaoka, M. 2007. Proc Natl Acad Sci USA 104:4095-4100.
12. Song, E., Zhu, P., Lee, S. K., Chowdhury, D., Kussman, S., Dykxhoorn, D. M., Feng, Y., Palliser, D., Weiner, D. B., Shankar, P. et al. 2005. Nat Biotechnol 23:709-717.
13. Mapara, M. Y., Kim, Y. M., Marx, J., and Sykes, M. 2003. Transplantation 76:297-305.
14. Wekerle, T., Kurtz, J., Ito, H., Ronquillo, J. V., Dong, V., Zhao, G., Shaffer, J., Sayegh, M. H., and Sykes, M. 2000. Nat Med 6:464-469.
15. Wekerle, T. 2001. J Heart Lung Transplant 20:816-823.
16. Leykin, I., Nikolic, B., and Sykes, M. 2001. Transplant Proc 33:120.
17. Nikolic, B., Takeuchi, Y., Leykin, I., Fudaba, Y., Smith, R. N., and Sykes, M. 2004. Diabetes 53:376-383.
18. Sykes, M. and Nikolic, B. 2005. Nature 435:620-627.
19. Bitko, V., Musiyenko, A., Shulyayeva, O., and Barik, S. 2005. Nat Med 11:50-55.
20. de Fougerolles, A., Vornlocher, H. P., Maraganore, J., and Lieberman, J. 2007. Nat Rev Drug Discov. 6:443-453.
21. Dykxhoorn, D. M. and Lieberman, J. 2006. Cell 126:231-235.
22. Fudaba, Y., Spitzer, T. R., Shaffer, J., Kawai, T., Fehr, T., Delmonico, F., Preffer, F., Tolkoff-Rubin, N., Dey, B. R., Saidman, S. L. et al. 2006. Am J Transplant 6:2121-2133.
23. Kawai, T., Cosimi, A. B., Spitzer, T. R., Tolkoff-Rubin, N., Suthanthiran, M., Saidman, S. L., Shaffer, J., Preffer, F. I., Ding, R., Sharma, V. et al. 2008. N Engl J Med 358:353-361.
24. Shaffer, J., Villard, J., Means, T. K., Alexander, S., Dombkowski, D., Dey, B. R., McAfee, S., Ballen, K. K., Saidman, S., Preffer, F. I. et al. 2007. Exp Hematol 35:1140-1152.
25. Tomita, Y., Sachs, D. H., and Sykes, M. 1994. Blood 83:939-948.
26. Ramshaw, H. S., Crittenden, R. B., Dooner, M., Peters, S. O., Rao, S. S., and Quesenberry, P. J. 1995. Biol. Blood Marrow Transplant. 1:74-80.
27. Sykes, M., Szot, G. L., Swenson, K., Pearson, D. A., and Wekerle, T. 1997. Exp. Hematol. 26:457-465.
28. Sykes, M., Szot, G. L., Swenson, K., and Pearson, D. A. 1997. Nature Med. 3:783-787.
29. Khan, A., Tomita, Y., and Sykes, M. 1996. Transplantation 62:380-387.
30. Manilay, J. O., Pearson, D. A., Sergio, J. J., Swenson, K. G., and Sykes, M. 1998. Transplantation 66:96-102.
31. Sykes, M. 2007. Front Biosci. 12:2922-2934.
32. Tomita, Y., Khan, A., and Sykes, M. 1994. J Immunol 153:1087-1098.
33. Kawai, T., Cosimi, A. B., COLVIN, R. B., Powelson, J., Eason, J., Kozlowski, T., Sykes, M., Monroy, R., Tanaka, M., and Sachs, D. H. 1995. Transplantation 59:256-262.
34. Kawai, T., Cosimi, A. B., Spitzer, T. R., Tolkoff-Rubin, N., Suthanthiran, M., Saidman, S. L., Shaffer, J., PREFFER, F. I., Ding, R., Sharma, V. et al. 2007. New Engl. J. Med. In press.
35. Kimikawa, M., Sachs, D. H., COLVIN, R. B., Bartholemew, A., Kawai, T., and Cosimi, A. B. 1997. Transplantation 64:709-716.
36. Mezrich, J. D., Yamada, K., Lee, R. S., Mawulawde, K., Benjamin, L. C., Schwarze, M. L., Maloney, M. E., Amoah, H. C., Houser, S. L., Sachs, D. H. et al. 2003. Transplantation 76:625-631.
37. Mezrich, J. D., Kesselheim, J. A., Johnston, D. R., Yamada, K., Sachs, D. H., and Madsen, J. C. 2003. Am J Transplant 3:1107-1115.
38. Menard, M. T., Schwarze, M. L., Allan, J. S., Johnston, D. R., Mawulawde, K., Shimizu, A., Yamada, K., Houser, S. L., Allison, K. S., Sachs, D. H. et al. 2004. Am J Transplant 4:79-86.
39. Bennett, S. R., Carbone, F. R., Karamalis, F., Flavell, R. A., Miller, J. F., and Heath, W. R. 1998. Nature 393:478-480.
40. Ridge, J. P., Di Rosa, F., and Matzinger, P. 1998. Nature 393:474-478.
41. Rifle, G. and Mousson, C. 2002. Transplantation 73:S1-S2.
42. Schoenberger, S. P., Toes, R. E., van der Voort, E. I., Offringa, R., and Melief, C. J. 1998. Nature 393:480-483.
43. Fehr, T., Takeuchi, Y., Kurtz, J., and Sykes, M. 2005. Eur J Immunol 35:2679-2690.
44. Kurtz, J., Ito, H., Wekerle, T., Shaffer, J., and Sykes, M. 2001. Am. J. Transplant. 1:339-349.
45. Kurtz, J., Shaffer, J., Anosova, N., Benichou, G., and Sykes, M. 2004. Blood 103:4336-4343.
46. Kim, D. H. and Rossi, J. J. 2007. Nat Rev Genet. 8:173-184.
47. John, M., Constien, R., Akinc, A., Goldberg, M., Moon, Y. A., Spranger, M., Hadwiger, P., Soutschek, J., Vornlocher, H. P., Manoharan, M. et al. 2007. Nature 449:745-747.
48. Judge, A. D., Bola, G., Lee, A. C., and MacLachlan, I. 2006. Mol Ther 13:494-505.
49. McCaffrey, A. P., Meuse, L., Pham, T. T., Conklin, D. S., Hannon, G. J., and Kay, M. A. 2002. Nature 418:38-39.
50. Song, E., Lee, S. K., Wang, J., Ince, N., Ouyang, N., Min, J., Chen, J., Shankar, P., and Lieberman, J. 2003. Nat Med 9:347-351.
51. Hadj-Slimane, R., Lepelletier, Y., Lopez, N., Garbay, C., and Raynaud, F. 2007. Biochimie 89:1234-1244.
52. Palliser, D., Chowdhury, D., Wang, Q. Y., Lee, S. J., Bronson, R. T., Knipe, D. M., and Lieberman, J. 2006. Nature 439:89-94.
53. Frederickson, R. M. 2007. Mol Ther 15:221-222.
54. Soutschek, J., Akinc, A., Bramlage, B., Charisse, K., Constien, R., Donoghue, M., Elbashir, S., Geick, A., Hadwiger, P., Harborth, J. et al. 2004. Nature 432:173-178.
55. Zimmermann, T. S., Lee, A. C., Akinc, A., Bramlage, B., Bumcrot, D., Fedoruk, M. N., Harborth, J., Heyes, J. A., Jeffs, L. B., John, M. et al. 2006. Nature 441:111-114.
56. Hu-Lieskovan, S., Heidel, J. D., Bartlett, D. W., Davis, M. E., and Triche, T. J. 2005. Cancer Res 65:8984-8992.
57. Heidel, J. D., Yu, Z., Liu, J. Y., Rele, S. M., Liang, Y., Zeidan, R. K., Kornbrust, D. J., and Davis, M. E. 2007. Proc Natl Acad Sci USA 104:5715-5721.
58. McNamara, J. O., Andrechek, E. R., Wang, Y., Viles, K. D., Rempel, R. E., Gilboa, E., Sullenger, B. A., and Giangrande, P. H. 2006. Nat Biotechnol 24:1005-1015.
59. Chen, S. Y., Khouri, Y., Bagley, J., and Marasco, W. A. 1994. Proc Natl Acad Sci USA 91:5932-5936.
60. Chen, S. Y., Zani, C., Khouri, Y., and Marasco, W. A. 1995. Gene Ther 2:116-123.
61. Huang, L., Shimaoka, M., Rondon, I. J., Roy, I., Chang, Q., Po, M., Dransfield, D. T., Ladner, R. C., Edge, A. S., Salas, A. et al. 2006. J Leukoc Biol 80:905-914.
62. Shimaoka, M., Kim, M., Cohen, E. H., Yang, W., Astrof, N., Peer, D., Salas, A., Ferrand, A., and Springer, T. A. 2006. Proc Natl Acad Sci USA 103:13991-13996.
63. Wekerle, T., Sayegh, M. H., Chandraker, A., Swenson, K. G., Zhao, Y., and Sykes, M. 1999. Transplant Proc 31:680.
64. Wekerle, T., Kurtz, J., Sayegh, M., Ito, H., Wells, A., Bensinger, S., Shaffer, J., Turka, L., and Sykes, M. 2001. J Immunol 166:2311-2316.
65. Ito, H., Kurtz, J., Shaffer, J., and Sykes, M. 2001. J. Immunol. 166:2981.
66. Kranz, D. M., Tonegawa, S., and Eisen, H. N. 1984. Proc Natl Acad Sci USA 81:7922-7926.
67. Sha, W. C., Nelson, C. A., Newberry, R. D., Kranz, D. M., Russell, J. H., and Loh, D. Y. 1988. Nature 335:271-274.
68. Davignon, D., Martz, E., Reynolds, T., Kurzinger, K., and Springer, T. A. 1981. J Immunol 127:590-595.
69. Davignon, D., Martz, E., Reynolds, T., Kurzinger, K., and Springer, T. A. 1981. Proc Natl Acad Sci USA 78:4535-4539.
70. Kurzinger, K., Reynolds, T., Germain, R. N., Davignon, D., Martz, E., and Springer, T. A. 1981. J Immunol 127:596-602.
71. Kurzinger, K. and Springer, T. A. 1982. J Biol Chem 257:12412-12418.
72. Ma, Q., Shimaoka, M., Lu, C., Jing, H., Carman, C. V., and Springer, T. A. 2002. J Biol Chem 277:10638-10641.
73. Sebastian, E. S., Matxain, J. M., Eriksson, L. A., Stote, R. H., Dejaegere, A., Cossio, F. P., and Lopez, X. 2007. J. Phys. Chem B 111:9099-9103.
74. Dreolini, L. and Takei, F. 2007. Biochem Biophys Res Commun 356:207-212.
75. Fagerholm, S. C., Hilden, T. J., Nurmi, S. M., and Gahmberg, C. G. 2005. J Cell Biol 171:705-715.
76. Kim, M., Carman, C. V., and Springer, T. A. 2003. Science 301:1720-1725.
77. Li, Y. F., Tang, R. H., Puan, K. J., Law, S. K., and Tan, S. M. 2007. J Biol Chem 282:24310-24319.
78. Simonson, W. T., Franco, S. J., and Huttenlocher, A. 2006 J Immunol 177:7707-7714.
79. Marlin, S. D. and Springer, T. A. 1987. (LFA-1). Cell 51:813-819.
80. Staunton, D. E., Marlin, S. D., Stratowa, C., Dustin, M. L., and Springer, T. A. 1988. Cell 52:925-933.
81. Staunton, D. E., Dustin, M. L., Erickson, H. P., and Springer, T. A. 1990. Cell 61:243-254.
82. Crucian, B., Neiman-Gonzalez, M., and Sams, C. 2006. Clin Vaccine Immunol 13:403-408.
83. Hogg, N., Henderson, R., Leitinger, B., McDowall, A., Porter, J., and Stanley, P. 2002. Immunol Rev 186:164-171.
84. Johnston, S. C., Dustin, M. L., Hibbs, M. L., and Springer, T. A. 1990. J Immunol 145:1181-1187.
85. Stanley, P. 1989. Mol Cell Biol 9:377-383.
86. Salas, A., Shimaoka, M., Phan, U., Kim, M., and Springer, T. A. 2006. J Biol Chem 281:10876-10882.
87. Essani, N. A., Fisher, M. A., Farhood, A., Manning, A. M., Smith, C. W., and Jaeschke, H. 1995. Hepatology 21:1632-1639.
88. Lan, P., Wang, L., Diouf, B., Eguchi, H., Su, H., Bronson, R., Sachs, D. H., Sykes, M., and Yang, Y. G. 2004. Blood 103:3964-3969.
89. Lan, P., Tonomura, N., Shimizu, A., Wang, S., and Yang, Y. G. 2006. Blood 108:487-492.
90. Oberdoerffer, P., Kanellopoulou, C., Heissmeyer, V., Paeper, C., Borowski, C., Aifantis, I., Rao, A., and Rajewsky, K. 2005. Mol Cell Biol 25:3896-3905.
91. Heffner, M. and Fearon, D. T. 2007. Proc Natl Acad Sci USA 104:13414-13419.
92. Kissler, S., Stern, P., Takahashi, K., Hunter, K., Peterson, L. B., and Wicker, L. S. 2006. Nat Genet. 38:479-483.
93. Goffinet, C. and Keppler, O. T. 2006. FASEB J 20:500-502.
94. McManus, M. T., Haines, B. B., Dillon, C. P., Whitehurst, C. E., van Parijs, L., Chen, J., and Sharp, P. A. 2002. J Immunol 169:5754-5760.
95. Roose, J. and Weiss, A. 2000. Nat Immunol 1:275-276.
96. Roose, J. P., Mollenauer, M., Gupta, V. A., Stone, J., and Weiss, A. 2005. Mol Cell Biol 25:4426-4441.
97. Roose, J. P., Mollenauer, M., Ho, M., Kurosaki, T., and Weiss, A. 2007. Mol Cell Biol 27:2732-2745.
98. Priatel, J. J., Chen, X., Zenewicz, L. A., Shen, H., Harder, K. W., Horwitz, M. S., and Teh, H. S. 2007. J Immunol 179:2143-2152.
99. Rowell, E. A. and Wells, A. D. 2006. Crit Rev Immunol 26:189-212.
100. Marrack, P. and Kappler, J. 2004. Annu Rev Immunol 22:765-787.
101. Desbiens, K. M., Deschesnes, R. G., Labrie, M. M., Desfosses, Y., Lambert, H., Landry, J., and Bellmann, K. 2003. Biochem J 372:631-641.
102. Nieminen, A. I., Partanen, J. I., and Klefstrom, J. 2007. Cell Cycle 6:2464-2472.
103. Nieminen, A. I., Partanen, J. I., Hau, A., and Klefstrom, J. 2007. EMBO J. 26:1055-1067.
104. Methi, T., Ngai, J., Mahic, M., Amarzguioui, M., Vang, T., and Tasken, K. 2005. J Immunol 175:7398-7406.
105. Tournier, C., Hess, P., Yang, D. D., Xu, J., Turner, T. K., Nimnual, A., Bar-Sagi, D., Jones, S. N., Flavell, R. A., and Davis, R. J. 2000. Science 288:870-874.
106. Yoshikawa, T., Uchimura, E., Kishi, M., Funeriu, D. P., Miyake, M., and Miyake, J. 2004. J Control Release 96:227-232.
107. Asaumi, N., Omoto, E., Mahmut, N., Katayama, Y., Takeda, K., Shinagawa, K., and Harada, M. 2001. Ann Hematol 80:387-392.
108. Peled, A., Grabovsky, V., Habler, L., Sandbank, J., Arenzana-Seisdedos, F., Petit, I., Ben Hur, H., Lapidot, T., and Alon, R. 1999. J Clin Invest 104:1199-1211.
109. Peled, A., Kollet, O., Ponomaryov, T., Petit, I., Franitza, S., Grabovsky, V., Slav, M. M., Nagler, A., Lider, O., Alon, R. et al. 2000. Blood 95:3289-3296.
110. Pruijt, J. F., van Kooyk, Y., Figdor, C. G., Willemze, R., and Fibbe, W. E. 1999. Blood 93:107-112.
111. Velders, G. A., Pruijt, J. F., Verzaal, P., van Os, R., van Kooyk, Y., Figdor, C. G., de Kruijf, E. J., Willemze, R., and Fibbe, W. E. 2002. Blood 100:327-333.
112. Colvin, G. A., Lambert, J. F., Dooner, M. S., Cerny, J., and Quesenberry, P. J. 2007. J Cell Physiol 211:386-391.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of inducing tolerance to a tissue or cell transplant in a subject, the method comprising administering to the subject
(a) a composition comprising a T-cell specific siRNA delivery reagent complexed with an siRNA that specifically induces anergy and death of activated T cells; and
(b) a hematopoietic stem cell transplant.

2. The method of claim 1, wherein the T-cell specific siRNA delivery reagent comprises (i) a fusion protein for delivery of a nucleic acid to activated T cells, wherein the fusion protein comprises:
a first portion comprising a T-cell targeting sequence that binds specifically to activated T cells; and
at least a second portion comprising a cationic sequence that electrostatically binds nucleic acid molecules.

3. The method of claim 1, wherein the T-cell specific siRNA delivery reagent comprises a nanoparticle, wherein the surface of the nanoparticle has attached thereto a T-cell targeting sequence and a cationic sequence that enables electrostatic binding of negatively charged siRNA molecules.

4. The method of claim 2, wherein the T-cell targeting sequence is selected from the group consisting of ICAM-1 or portions thereof, or antibodies or antigen-binding portions thereof that specifically bind to the HA conformation of LFA-1, CD69, CD25, CD44, ICOS, or an activated T-cell specific cytokine receptor.

5. The method of claim 4, wherein the antigen-binding portions are scFV, Fab, or Fab′2.

6. The method of claim 2, wherein the cationic sequence that enables electrostatic binding of negatively charged siRNA molecules comprises human protamine or a cationic nucleic acid-binding portion thereof.

7. The method of claim 2, wherein the fusion protein further comprises a secretion signal peptide that promotes secretion from the cell.

8. The method of claim 2, wherein the fusion protein further comprises a multimerization domain.

9. The method of claim 8, wherein the multimerization domain comprises IgG Fc having at least an immunoglobulin CH2 and CH3 domain.

10. The method of claim 2, wherein the fusion protein further comprises a linker between the first and second portions.

11. The method of claim 2, wherein the fusion protein further comprises a protein purification sequence.

12. The method of claim 11, wherein the protein purification sequence is His6 or an Fc region.

13. The method of claim 1, wherein the siRNA specifically targets a gene encoding a protein selected from the group consisting of RasGRP1, cyclin D1, and bcl-xL include bcl-2, mcl-1, Akt, N-ras, SOS, Zap70, mTOR, NFAT, NFkB, polo-like kinases (plk), cFLIP, and ICAD.

14. The method of claim 1, further comprising transplanting a tissue or organ into the subject.

15. (canceled)
